(19) United States
(12) Patent Application Publication
(10) Pub. No.: US 2026/0363607 A1
(43) Pub. Date: Nov. 19, 2026

(54) WEARABLE SENSOR FOR MONITORING SOLID AND LIQUID CONSUMPTION

(71) Applicant: Eli Lilly and Company, Indianapolis, IN (US)

(72) Inventors: Tae Hoo CHANG, Michuhol-gu, Incheon (KR); Eyal DASSAU, Acton, MA (US); Min Ku KIM, Seoul (KR); Chi Hwan LEE, West Lafayette, IN (US); Taewoong PARK, West Lafayette, IN (US); Candace J. SCHERER, Indianapolis, IN (US)

(21) Appl. No.: 19/121,024

(22) PCT Filed: Oct. 12, 2023
(86) PCT No.: PCT/US2023/076671

§ 371 Date: Apr. 14, 2025

Related U.S. Application Data
(60) Provisional application No. 63/416,027, filed on Oct. 14, 2022.

(51) Int. Cl. A61B 5/00; A61B 5/397

(52) U.S. Cl. CPC A61B 5/4205; A61B 5/397; A61B 5/6822

(57) ABSTRACT

The present disclosure relates to a wearable device for monitoring food and drink intake of a wearer for creation of a consumption timeline of a wearer. The wearable device may make a distinction between food and drink consumption for greater reliability, which may increase effectiveness of patient care adjustment in relation to food-related afflictions. The wearable device may also be self-contained so that the user is not required to carry or keep track of a separate receiver for collection and processing of said consumption information.

### FIELD OF THE DISCLOSURE

The present disclosure relates to a wearable device for the monitoring of muscle activity. In particular, the present disclosure relates to a wearable sensor for the monitoring of throat muscle activity, which may be related to consumption of liquids or solids.

### BACKGROUND OF THE DISCLOSURE

Common food-related afflictions often require the monitoring of patient food and drink consumption. Such monitoring allows for adjustment of such consumption when needed, or may allow for adjustment of treatment, including a change in dosage of therapeutic agents, to properly treat the patient in view of such consumption. For example, for obese patients or patients suffering from diabetes, food monitoring may help! the patient create awareness around their individual food consumption or provide a caretaker with information for adjustment of insulin treatment.

Unfortunately, such monitoring often relies on self-reporting by the patient, which is often an unreliable method of keeping a food journal, as food and drink consumption may be over or underreported, whether intentionally or unintentionally. As such, a more reliable source of information is needed to allow for reliable reporting, resulting in confident care adjustment and effective treatment.

### SUMMARY

The present disclosure relates to a wearable device for monitoring food and drink intake of a wearer for creation of a consumption timeline of a wearer. The wearable device may make a distinction between food and drink consumption for greater reliability, which may increase effectiveness of patient care adjustment in relation to food-related afflictions. The wearable device may also be self-contained so that the user is not required to carry or keep track of a separate receiver for collection and processing of said consumption information. The wearable device may be a medical device or a lifestyle or wellness device. Such a device, as well as the automated consumption timeline discussed herein, may be used for a variety of purposes, such as early detection and diagnosis of eating disorders, early diagnosis of conditions that may lead to obesity prior to increase in Body Mass Index (BMI), and/or to aid in delivery of medications such as insulins whose dosage and/or time of administration may need to be adjusted based on times and amount of food and drink consumption (e.g., as part of an automated insulin delivery system).

In some embodiments, instead of (or in addition to) monitoring food and drink intake of the wearer, the presently disclosed wearable device may also be used to detect vomiting or emesis. This vomiting or emesis may, but need not be, related to eating disorders or other types of illness or conditions. For example, the presently disclosed device may be used to detect vomiting or emesis by critically ill patients, elderly patients, and/or young babies.

In some embodiments, a wearable device is provided. The wearable device comprises a textile and a sensor system affixed to the textile, the sensor system comprising at least two sEMG recording electrodes, an accelerometer, a circuit signal amplifier, a microcontroller, and a memory. At least two sEMG recording electrodes and the accelerometer of the sensor system may be configured to be positioned over a thyrohyoid muscle of a human subject (e.g., a human user or individual). The wearable device may be configured for measuring data associated with underlying thyrohyoid muscle activity suitable for detecting a physical event of the human subject. Examples of such physical events include eating events (including chewing and swallowing) and drinking events.

In other embodiments, a consumption journaling system is provided. The system may comprise a wearable device having a sensor system comprising at least two sEMG recording electrodes, an accelerometer, and a circuit signal amplifier. The sensor system may be configured to measure underlying muscle activity when the wearable device is positioned on a neck of a human subject. The system may also comprise a processor configured to analyze the measured underlying muscle activity to detect and distinguish between a solid food consumption event and a drink consumption event and log the solid food consumption event or the drink consumption event when detected.

In yet other embodiments, a method for creating a consumption timeline is provided. The method may comprise positioning a wearable device on a thyrohyoid muscle of a human subject, measuring thyrohyoid muscle activity with a plurality of sensors of the wearable device, processing the measured thyrohyoid muscle activity, identifying a physical event according to the processed thyrohyoid muscle activity, wherein the physical event is differentiated as one of a food consumption event and a non-food consumption event, and recording the physical event according to a timeline when the physical event is a food consumption event.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 illustrates a wearable device of the present disclosure, wherein the wearable device includes a sensor system affixed to a textile which may be worn on a user's neck;

FIG. 2 is an exploded view of the wearable device of FIG. 1, including the textile, a base, an electronics and sensor assembly, and a cover;

FIG. 3A is a schematic view of the electronics and sensor assembly of the wearable device of FIG. 2;

FIG. 3B is a block diagram of a computing system including a network, a remote computing device, and the wearable device of FIG. 2;

FIG. 4 is a schematic view of a signal amplifier circuit of the electronics and sensor assembly of FIG. 3A;

FIG. 5 is a schematic view of a sensor system of a wearable device of the present disclosure;

FIG. 6A is an exploded perspective view of the wearable device including the sensor system of FIG. 5 in a stacked configuration relative to a user's neck;

FIG. 6B is a perspective and side view of the wearable device of FIG. 6A encapsulated in a polymer and including a precurved surface.

FIG. 6C is a schematic view of a user's neck with the wearable device of FIG. 6B positioned thereon;

FIG. 7 is an exploded side view of the wearable device of FIG. 6A;

FIG. 8 is a chart of a method for operation of a wearable device for creation of a consumption journal;

FIG. 9A illustrates a response of a signal amplifier circuit in response to a series of swallowing events;

FIG. 9B illustrates sEMG measurements in response to a series of swallowing events;

FIG. 9C illustrates a Fourier transform of the sEMG measurements of FIG. 9B in response to the series of swallowing events;

FIG. 10A illustrates a measured amplitude of sEMG measurements at a sampling rate of 1 kHz in response to a series of swallowing events;

FIG. 10B illustrates a measured amplitude of sEMG measurements at a sampling rate of 500 Hz in response to a series of swallowing events;

FIG. 10C illustrates a measured amplitude of sEMG measurements at a sampling rate of 250 Hz in response to a series of swallowing events;

FIG. 10D illustrates a measured amplitude of sEMG measurements at a sampling rate of 125 Hz in response to a series of swallowing events;

FIG. 11A illustrates various possible positions of sEMG recording electrodes for measuring of underlying muscle activity, including a first position, a second position, and a third position;

FIG. 11B illustrates a comparison of sEMG measurements during a swallowing event between the first position, the second position, and the third position of FIG. 11A;

FIG. 12 illustrates muscle activity as measured by sEMG recording electrodes and an accelerometer over a given time period during which a subject remains stationary;

FIG. 13 illustrates muscle activity as measured by sEMG recording electrodes and an accelerometer over a given time period during which a subject is walking;

FIG. 14 illustrates muscle activity as measured by sEMG recording electrodes and an accelerometer over a given time period during which a subject is talking;

FIG. 15 illustrates muscle activity as measured by sEMG recording electrodes and an accelerometer over a given time period during which a subject is eating solid food;

FIG. 16A illustrates muscle activity as measured by sEMG recording electrodes, an accelerometer, and a microphone over a given time period during which a subject is remaining stationary;

FIG. 16B illustrates a series of two-dimensional spectrograms consistent with the muscle activity of FIG. 16A;

FIG. 16C illustrates a series of three-dimensional spectrograms consistent with the muscle activity of FIG. 16A;

FIG. 17A illustrates muscle activity as measured by sEMG recording electrodes, an accelerometer, and a microphone over a given time period during which a subject is speaking;

FIG. 17B illustrates a series of two-dimensional spectrograms consistent with the muscle activity of FIG. 17A;

FIG. 17C illustrates a series of three-dimensional spectrograms consistent with the muscle activity of FIG. 17A;

FIG. 18A illustrates muscle activity as measured by sEMG recording electrodes, an accelerometer, and a microphone over a given time period during which a subject is walking;

FIG. 18B illustrates a series of two-dimensional spectrograms consistent with the muscle activity of FIG. 18A;

FIG. 18C illustrates a series of three-dimensional spectrograms consistent with the muscle activity of FIG. 18A;

FIG. 19A illustrates muscle activity as measured by sEMG recording electrodes, an accelerometer, and a microphone over a given time period during which a subject is eating solid food, including chewing and swallowing such food;

FIG. 19B illustrates a series of two-dimensional spectrograms consistent with the muscle activity of FIG. 19A;

FIG. 19C illustrates a series of three-dimensional spectrograms consistent with the muscle activity of FIG. 19A;

FIG. 20A illustrates muscle activity as measured by sEMG recording electrodes, an accelerometer, and a microphone over a given time period during which a subject is drinking;

FIG. 20B illustrates a series of two-dimensional spectrograms consistent with the muscle activity of FIG. 20A;

FIG. 20C illustrates a series of three-dimensional spectrograms consistent with the muscle activity of FIG. 20A;

FIGS. 21A-21D illustrate distinguishing graphical features of measured muscle activity, acceleration, and sound pressure waves for walking, talking, swallowing, chewing, and drinking;

FIG. 22 illustrates a graphical delineation of events with corresponding sEMG signal amplitudes and acceleration frequency;

FIG. 23A illustrates the strain of a bridge section of a sensor module of a wearable device of the present disclosure;

FIG. 23B illustrates the strain of a sensor module of a wearable device of the present disclosure;

FIG. 23C illustrates the strain of sensor modules having varying precurved designs on varying curvatures of human skin;

FIG. 23D illustrates the mechanical durability of the wearable device under conditions of 60% stretching and simultaneous twisting;

FIG. 23E illustrates the maximum strain variations within the sensor module under the conditions of FIG. 23D;

FIG. 23F illustrates the mechanical assessments for sensor modules in two distinct sizes when subjected to a 60% stretch;

FIG. 23G illustrates the mechanical assessments for sensor modules in two distinct sizes when subjected to a 120% stretch;

FIG. 24 illustrates hemoglobin contents multiplied by the optical pathlength (b₄) to indicate skin irritation;

FIG. 25A illustrates vibration recording over time for sensor modules in two distinct sizes;

FIG. 25B illustrates a fast Fourier transform (FFT) analysis of the results of FIG. 25A;

FIG. 25C illustrates sustained functionality of a wearable device of the present disclosure under a continuous flow of salt water;

FIG. 25D illustrates pressure maintenance of precurved and non-precurved sensor modules;

FIG. 25E illustrates the temperature increase of sensor modules over 18 hours of wear time;

FIG. 25F illustrates measurement results of average hemoglobin contents of the skin;

FIG. 26 illustrates temperature variations on a sensor module surface and adjacent skin areas over an 18-hour period;

FIG. 27A illustrates a stationary experiment scenario wherein a subject engages in various activities;

FIG. 27B illustrates signals recorded from various sensors of a sensor module during the various activities of FIG. 27A over time;

FIG. 27C illustrates a granular view of the specific signal patterns associated with the various activities of FIG. 27A;

FIG. 28A illustrates an experiment scenario wherein a subject engages in various activities while simultaneously walking;

FIG. 28B illustrates signals recorded form various sensors of a sensor module during the various activities of FIG. 28A over time;

FIG. 28C illustrates a granular view of the specific signal patterns associated with the various activities of FIG. 28A;

FIG. 29A illustrates a model for recognizing activity using a sensor module;

FIG. 29B illustrates a majority ruling method for the model of FIG. 29A;

FIG. 29C illustrates classification of activities using the models and methods of FIGS. 29A and 29B;

FIG. 30A illustrates a signal processing pipeline for the optimization of RF classifier parameters for the classification model of the present disclosure;

FIG. 30B illustrates the performance for accuracy over number of trees using the classification model of the present disclosure and optimization of RF classifier parameters of FIG. 30A;

FIG. 30C illustrates the performance for accuracy over frame length using the classification model of the present disclosure and optimization of RF classifier parameters of FIG. 30A;

FIG. 30D illustrates the performance for accuracy over frame number using the classification model of the present disclosure and optimization of RF classifier parameters of FIG. 30A;

FIGS. 30E-30F illustrate the labels and corresponding accuracy of identified individual and concurrent activities according to the classification model of the present disclosure and optimization of RF classifier parameters of FIG. 30A;

FIG. 30G illustrates a confusion matrix including accuracy attained for each activity during concurrent activities according to the classification model of the present disclosure and optimization of RF classifier parameters of FIG. 30A;

FIG. 30H illustrates a counterpart matrix to the confusion matrix of FIG. 30G for individual activities;

FIG. 30I illustrates the input signals of individual sensor module components during individual and concurrent activities; and

FIG. 30J illustrates a comparative analysis between conventional classification models and the classification model of the present disclosure.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

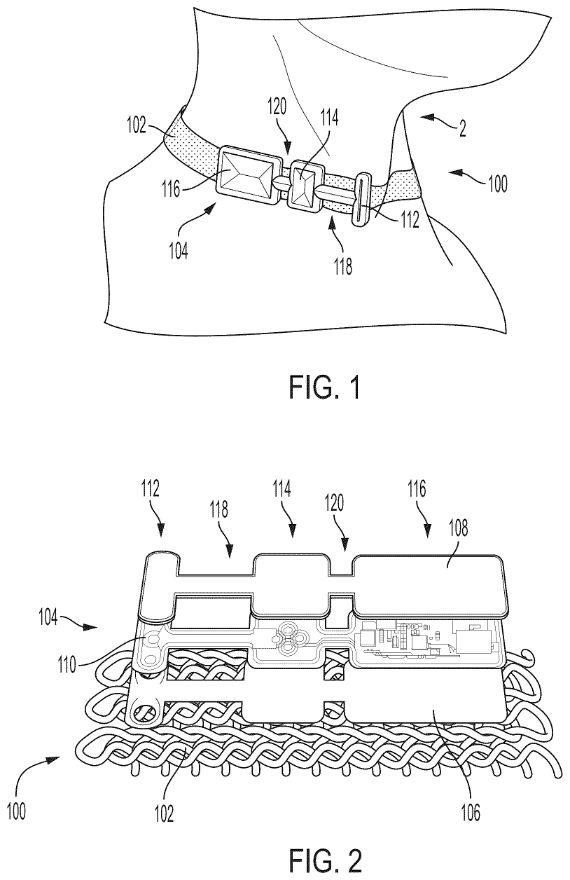

Referring to FIG. 1, a wearable device 100 is illustrated. The wearable device 100 may include a textile 102 for positioning on or around a human subject's neck 2 and a sensor system 104 affixed to the textile 102. As shown, the textile 102 is configured for placement encircling the human subject's neck 2, which may allow for proper positioning of the sensor system 104 in an unobtrusive manner. In other embodiments, the textile 102 and/or the sensor system 104 may be removably positioned on the human subject's neck 2 using other methods, such as, for example, body-safe adhesive. The textile 102 may be a stretchable and recoverable textile that allows for a comfortable but proper fit on the human subject's neck 2 for up to at least 24 hours. The textile may be breathable and formed of, for example, cotton, polyester, nylon, rayon, linen, silk, micromodal (high-wet modulus rayon), merino wool, chambray, and any combinations thereof.

Referring additionally to FIG. 2, the sensor system 104 of the wearable device may include a base 106 and a cover 108 with an electronics and sensor assembly 110 positioned between the base 106 and the cover 108. The base 106 and/or the cover 108 may be formed of a flexible polymer or polymer composite, such as, for example, EcoFlex®, Silbione™, Sylgard® 184, and the like. Such flexibility allows for the base 106 and cover 108 to conform to the shape of the human subject's neck 2 and, correspondingly, the underlying textile 102 without sacrificing protection to the interior electronics and sensor assembly 110. The electronics and sensor assembly 110 may comprise, be disposed on, or be formed out of, a flexible electronic substrate that may also conform to the shape of the human subject's neck 2.

As illustrated, the sensor system 104 may include a first segment 112, a second segment 114, and a third segment 116 as described further herein. Each of the segments 112, 114, and 116 are shaped and sized for desired positioning of each segment relative to the underlying human subject's neck 2 and containment of respective electronics and sensors as described further herein. For instance, the sensor system 104 may be positioned over a thyrohyoid muscle of the neck 2. A first bridge section 118 may physically couple the first segment 112 with the second segment 114 and provide a conduit for electrical connection between the first segment 112 and the second segment 114. A second bridge section 120 may physically couple the second segment 114 and the third segment 116 and provide a conduit for electrical connection between the second segment 114 and the third segment 116. Each of the first bridge section 118 and the second bridge section 120 may be narrower in width relative to each of the first segment 112, the second segment 114, and the third segment 116 to increase flexibility of the sensor system 104 to facilitate wrapping of the sensor system 104 around the human subject's neck 2 with the corresponding underlying textile 102 and/or base 106. In other embodiments, the first segment 112, the second segment 114, and the third segment 116 may be distinct from each other without the first bridge section 118 and/or the second bridge section 120 resulting in a physical coupling, although the first segment 112, the second segment 114, and the third segment 116 may be otherwise operationally coupled (e.g., such as by wireless communications).

While the sensor system 104 is illustrated as including three segments, in other embodiments, the first segment 112, the second segment 114, and the third segment 116 and the components thereof may be consolidated into one or two segments. In yet other embodiments, the first segment 112, the second segment 114, and the third segment 116 and their respective components may be further divided into a greater number of segments. The respective components of each of the first segment 112, the second segment 114, and the third segment 116 as described further herein may be otherwise arranged.

As shown in FIG. 1, placement of the sensor system 104 positions the first segment 112 over the thyrohyoid muscle of the human subject's throat. In other embodiments, the first segment 112 may be positioned over the mylohyoid muscle, the sternothyroid muscle, the sternohyoid muscle, the omohyoid muscle, the stylohyoid muscle, or the digastric muscle. The second segment 114 and the third segment 116 may wrap around the human subject's neck 2 with positioning of the underlying textile 102.

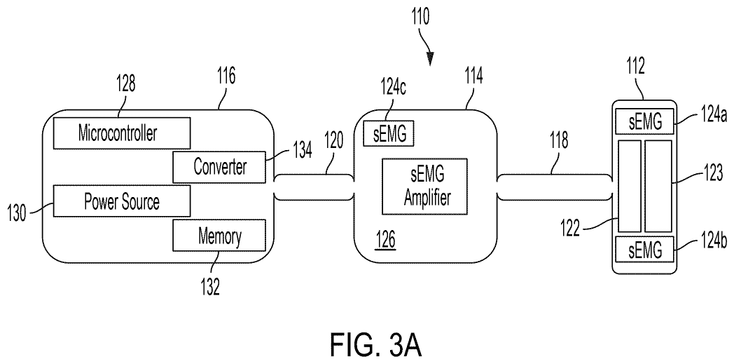

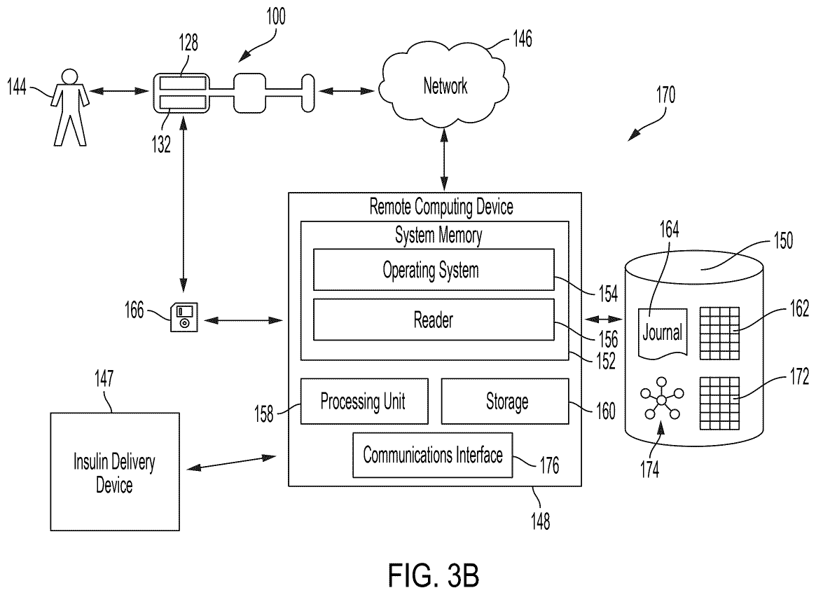

Now referring to FIG. 3A, a schematic illustration of the electronics and sensor assembly 110 of sensor system 104 (FIG. 2) is provided. As illustrated, the first segment 112 may house an accelerometer 122, a microphone 123, and at least two surface electromyogram (sEMG) recording electrodes 124a, 124b. The second segment 114 may house a reference recording sEMG electrode 124c and a signal amplifier circuit 126, which may be used for amplification of the signals generated by the sEMG recording electrodes 124a, 124b, 124c. The third segment 116 may house a microcontroller 128, a power source 130, a memory 132, and/or one or more analog to digital converters (ADC) 134. In some embodiments, a separate ADC converter may be provided for each type of signal generated by the sEMG recording electrodes 124a, 124b, and 124c, as well as microphone 123 and accelerometer 122. The microcontroller 128 may process muscle activity data recorded by sEMG recording electrodes 124a, 124b, 124c and accelerometer 122, which may include, for example, converting sensor data from the sEMG and/or the accelerometer into data representative of muscle activity in volts (V), acceleration (m/s² or g), hertz (Hz), and/or other suitable units. In further aspects, the microcontroller 128 may analyze the processed muscle activity data (processed data) to identify at least one corresponding physical event consistent with the muscle activity data. The power source 130 may include any power source capable of providing operational power to the various electronics and sensors within the electronics and sensor assembly 110, for example, a rechargeable battery capable of powering the wearable device 100 (FIG. 1) for at least 24 hours. The power source 130 may be a lithium-ion battery. In some embodiments, the power source 130 may be any other battery capable of powering the wearable device 100 for a time period less than or more than 24 hours. In some embodiments, the battery may be rechargeable.

The memory 132 may be a memory such as a micro- or mini-secure digital memory card which may be selectively received by the wearable device 100 (FIG. 1) and selectively removed from the wearable device 100 (FIG. 1) for further processing and/or review by a remote computing device. In other embodiments, the memory 132 may be an integrated, on-chip memory accessible via wireless transmission or selectively attachable wiring to a remote computing device for further processing and/or review. The memory 132 is preferably a non-volatile memory capable of receiving and preserving data through a plurality of start-ups and shut-downs of the electronics and sensor assembly 110.

The sEMG recording electrodes 124a, 124b, 124c, the accelerometer 122, and the microphone 123 may cooperate to measure and monitor movement of the underlying muscle, i.e., the thyrohyoid muscle, as described further herein. For example, sEMG recording electrodes 124a, 124b, 124c are capable of measuring muscle function from the skin surface above the muscle. In particular, the sEMG recording electrodes 124a, 124b, 124c can measure the voltage (e.g., electric potential) generated across the underlying muscle cells when said muscle cells are activated. The voltages measured by each electrode are then compared to determine differences in electric potential across the underlying muscle cells, creating an electromyogram to map the underlying muscle activity. Similarly, an accelerometer 122 and/or a microphone 123 is/are capable of measuring the contraction of the underlying muscle, offering additional data related to muscle activity. The cooperation of the sEMG recording electrodes 124a, 124b, 124c, the accelerometer 122, and microphone 123 may be utilized to detect underlying muscle function which can then be utilized to identify individual physical events in which the human subject is participating. For example, the sEMG recording electrodes 124a, 124b, 124c, the accelerometer 122, and the microphone 123 may cooperate to detect body movement, body orientation, acoustical vibration of the voice through the skin, and muscle activity during daily activities, including, for example, body movement, speaking, chewing, and swallowing.

As described further herein, the signal amplifier circuit 126, the microcontroller 128, and the analog to digital converter 134 may cooperate to receive and process the muscle activity data generated by the sEMG recording electrodes 124a, 124b, 124c, accelerometer 122, and microphone 123 for storing within the memory 132 for further processing. In some aspects, the muscle activity data may be stamped with a current time and stored as raw data in memory 132. In some aspects, initial processing, measurement, and storage may occur within self-contained wearable device 100 (FIG. 1) without the need for a transmitter and remote receiver for processing of data.

Now referring to FIG. 3B, a block diagram of a computing system including a network, a remote computing device, and the wearable device 100 of FIG. 1 is provided. As described above, memory 132 (FIG. 3A) may be a micro- or mini-secure digital memory card which may be selectively removed from the wearable device 100 (FIG. 1) for further processing and/or review by a remote computing device. In other embodiments the memory 132 may be an integrated, on-chip memory accessible via wireless transmission or selectively attachable wiring to a remote computing device for further processing and/or review. The memory 132 is preferably a non-volatile memory capable of receiving and preserving data through a plurality of start-ups and shut-downs of the electronics and sensor assembly 110 (FIG. 3A). In further aspects, as described above, the microcontroller 128 may be responsible for at least some processing of the muscle activity data. However, further processing and analysis may be performed by a processing unit 158 associated with remote computing device 148.

As illustrated by FIG. 3B, computing system 170 includes the wearable device 100 (FIG. 1), a remote computing device 148, a network 146, and a database 150. According to some aspects described above, FIG. 3A illustrates a computing system 170 for remote processing and analyses of the muscle activity data. In a basic configuration, the wearable device 100 monitors and records muscle activity of user 144 (e.g., human subject). In some examples described above, memory 132 may be a digital memory card (e.g., digital memory card 166), which may be selectively removed from the wearable device 100 (FIG. 1) and associated with a reader 156 via a driver of the remote computing device 148. In aspects, reader 156 may extract the muscle activity data from the digital memory card 166 for further processing and/or analyses by remote computing device 148. In other examples described above, memory 132 may be an integrated, on-chip memory accessible via wireless transmission over network 146 through communications interface 176 for further processing and/or analyses by remote computing device 148. In yet other examples, memory 132 may be accessible directly by remote computing device 148 via wireless transmission, without the necessity of any intervening network 146.

In aspects, remote computing device 148 may comprise a mobile device (e.g., a smartphone, or wearable device such as a smartwatch) that communicates directly with device 100 via wireless transmission, or a server that communicates with device 100 solely via a network 146 such as a cellular or data network. Remote computing device 148 may also comprise, or be communicatively coupled to, a medical device, such as an insulin pump or glucose monitoring device, such as a Continuous Glucose Monitor (CGM), Flash Glucose Monitor (FGM), and/or Blood Glucose Monitor (BGM). Remote computing device 148 may include at least one processing unit 158 and a system memory 152. In some aspects, the processing unit 158 may process muscle activity data received from the wearable device 100, which may include, for example, converting sensor data from the sEMG 126, the accelerometer 122, and/or the microphone 123 into data representative of muscle activity in volts (V) and/or hertz (Hz), respectively. In further aspects, the processing unit 158 may analyze the processed muscle activity data (processed data) to identify at least one corresponding physical event consistent with the muscle activity data. This analysis may be performed, for example, using a machine-learning algorithm that is trained to associate data patterns with corresponding physical events for automatically identifying physical events corresponding to the muscle activity data, including distinguishing the identified physical events between food-consumption and non-food-consumption events. In some aspects, other methods may be used to identify corresponding physical events based on the processed data, for example, by comparing newly processed data to previously processed data corresponding to identified physical events.

In some aspects, the processing unit 158 may execute logic for implementing a consumption journaling system. The identified physical events may include a variety of food-consumption or non-food-consumption events, including, for example, body orientation, walking, running, talking, sitting down, head and jaw movement, and other physical events that may require or use movement or other activity of the muscles (e.g., thyrohyoid muscles) underlying the wearable device 100. In aspects, the processing unit 158 may log a date, time, and/or duration of each food-consumption event (e.g., eating or drinking event) over a monitored time period to create a consumption journal. Such consumption journal may then be stored in storage 160 and/or database 150, for example. As noted above, obese or diabetic patients are often asked to manually record eating and/or drinking consumption, which may cause inaccurate records. As a benefit of the disclosed system, the consumption journal may be used by human subjects to supplement a food diary, log carbohydrate intake to better determine insulin dosage, or become more mindful of when and how much they consume (whether food or drink). Such a consumption journal may also be used in clinical trials in which accurate records of food and drink consumption would be helpful in measuring therapeutic effects of drug or non-drug intervention (e.g., for obesity or diabetes drugs or other interventions). Still further, such a consumption journal may be used by a provider to instruct a human subject to adjust food, liquids, or medication for food-related illnesses, such as obesity or diabetes, or in preparation for surgery to reduce potential complications.

The processing unit 158 may also execute logic for implementing a feed forward alert event that alerts or reminds the user to take insulin to counteract the food and/or drink that he/she just consumed. For example, such an alert may be delivered to a user via a user-interface within, or communicatively coupled to, remote computing device 148. For example, such an alert may be delivered as part of a push notification on the user's mobile device or smartwatch, or as an audible or visual indication delivered by a user's CGM. The push notification may also remind the user to provide the type of food or drink consumed, and/or an estimated amount of carbohydrates (or other macronutrients) consumed. Such alerts or reminders, or automated delivery of insulin, may help persons with diabetes to improve management of their Type 1 or Type 2 diabetes.

Beyond simply alerting or reminding a user of a food and/or drink consumption event, processing unit 158 may also determine at least one of a recommended dosage amount and a recommended dosage time for an administration of insulin based on the recorded food and/or drink consumption event. For example, when processing unit 158 detects a food or drink consumption event, the processing unit 158 may automatically calculate an amount of insulin that it estimates should be sufficient to counteract an expected rise in the user's glucose level as a result of the consumption event. The amount of insulin may be based on an estimated typical amount of carbohydrates that the user typically consumes in a single consumption event. Alternatively, or in addition, the amount of insulin may be further tailored based on the duration of the consumption event (e.g., a longer duration may indicate that more carbohydrates were consumed) and/or whether the consumption event is a food consumption event or a drink consumption event (e.g., a food consumption event may indicate the user consumed more carbohydrates compared to a drink consumption event). Alternatively or in addition, the amount of insulin may be tailored based on an amount of carbohydrates that the user provided in response to the aforementioned push notification. Alternatively or in addition, processing unit 158 may determine a recommended dosage time, e.g., by adjusting a scheduled time for a next administration of insulin to an earlier time based on the detected consumption event. The recommended dosage amount and/or recommended dosage time may be presented to the user via a user-interface within, or communicatively coupled to, remote computing device 148. Alternatively, or in addition, the recommended dosage amount and/or time may be used instruct an optional automated insulin delivery device 147 to administer insulin according to, or based on, the recommended dosage amount and/or dosage time. The aforementioned may be particularly useful in embodiments where remote computing device 148 comprises or is communicatively coupled to a medical device such as an insulin pump or any of the aforementioned glucose monitoring devices (e.g., CGM, FGM, or BGM).

Depending on the configuration and type of the remote computing device 148, the system memory 152 may include, but is not limited to, volatile storage (e.g., random access memory), non-volatile storage (e.g., read-only memory), flash memory, or any combination of such memories. The system memory 152 may also include an operating system 154, which may be suitable for controlling the operation of the remote computing device 148. The remote computing device 148 may also have additional data storage 160 for storing muscle activity data received from the wearable device 100, for example. As further illustrated by FIG. 3B, the remote computing device 148 may communicate with a database 150. Database 150 may comprise additional external storage that may be capable of storing muscle activity data 162, training data 172, neural network 174, and/or the consumption journal 164, for example.

In aspects, training data 172 may be created using muscle activity data 162 stamped with a current time (e.g., received from memory 132 of the wearable device 100) and manually correlated with an identified physical event at the same time. The manually correlated physical event may include, for example, food-consumption related events, non-food-consumption related events, swallowing events, chewing events, walking events, talking events, and the like. In further examples, neural network 174 may be trained using the training data 172 to produce a trained machine-learning algorithm for automatically identifying physical events associated with real-time measured muscle activity data. Further, the trained machine-learning algorithm may automatically distinguish between non-food-consumption related events and food-consumption related events, as well as distinguishing food-consumption events associated with talking, chewing, or swallowing, for instance. The output of the trained machine-learning algorithm may be used by the consumption journaling system to automate handwritten journaling, for example.

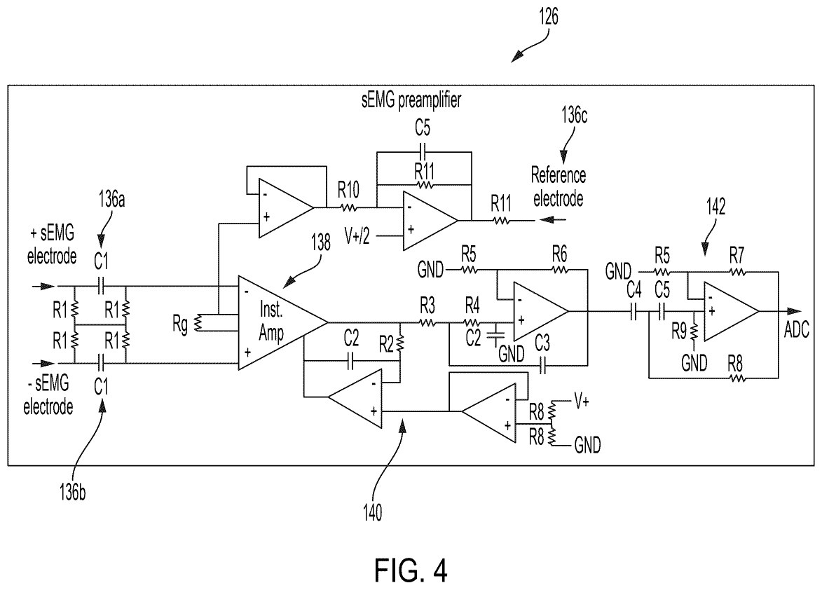

A schematic view of the signal amplifier circuit 126 is provided in FIG. 4. As shown, the signal amplifier circuit 126 may include an alternating current (AC) input coupling 136a, 136b for each of the sEMG recording electrodes 124a, 124b, and an additional input coupling 136c for reference sEMG recording electrode 124c to act as a ground for the recording electrodes. In some aspects, the sEMG recording electrodes 124a, 124b, 124c may be capable of capturing signals ranging from 0.1 Hz to 800 Hz. However, it may be desirable to filter out both low frequency and high frequency noise from the recorded sEMG signals before analyzing the signals for eating and/or drinking events.

Specifically, it may be desirable to filter out signals with frequencies less than 10 Hz. For example, such sEMG signals with frequencies less than 10 Hz may be less useful for detecting swallowing and/or drinking because it may include noise introduced by motion artifacts caused by gross body movements, such as walking, standing-up, and/or sitting-down, etc. In addition, filtering out low frequencies may also be helpful in filtering out any direct current (DC) offset in the sEMG signals. Filtering out such DC offsets may be particularly helpful in decreasing the probability that the filtered signal would exceed the measurable voltage range of the sensor device 104 (e.g., saturate the sensor device 104). Since the frequency band required for analysis may be narrowed further using software filters, the hardware filter may be configured to pass through frequencies over a relatively wide bandwidth to ensure no meaningful information is inadvertently filtered out. It may also be helpful to filter out signals with frequencies greater than 500 Hz. This is so because, in some embodiments, the signal frequencies useful for detecting eating and drinking events typically occur around 100 Hz, and thus frequencies greater than 500 Hz may be considered unhelpful noise.

For these reasons, the signals output by the sEMG electrodes may be filtered using a high-pass filter with a 10 Hz minimum threshold, and a low-pass filter with a 500 Hz maximum threshold. This is shown in FIG. 4, which includes an adjustable gain instrumentation amplifier 138, a second order Butterworth response high pass filter 140 with a frequency threshold of fc=10 Hz, and a low pass filter 142 with a frequency limit of fc=500 Hz.

While the embodiment described above in relation to FIG. 4 is designed to filter out signals above 500 Hz and below 10 Hz, it should be appreciated that other embodiments may use different threshold frequencies. For instance, if the sensor device 104 is powered by a voltage source that accommodates higher voltages, the minimum threshold of the high-pass filter may be adjusted accordingly. For instance, the minimum threshold may be about 5 Hz, 10 Hz, 20 Hz, or the like. Similarly, to ensure appropriate capture of all frequencies of interest, the maximum threshold may be adjusted in different embodiments to include higher frequencies, such as 600 Hz, 700 Hz, 900 Hz, 1000 Hz, or any value above, below or between. The threshold frequencies of both the high-pass filter 140 and the low-pass filter 142 may be adjusted by adjusting the values for the resistors and capacitors shown in FIG. 4.

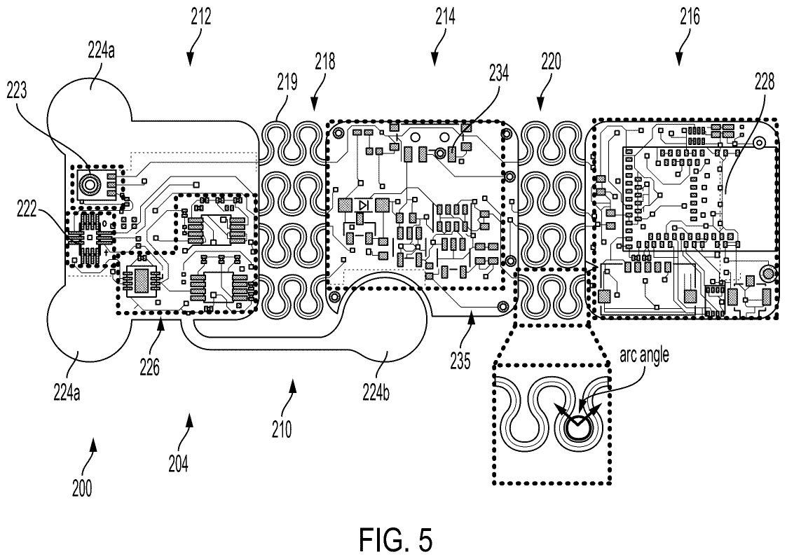

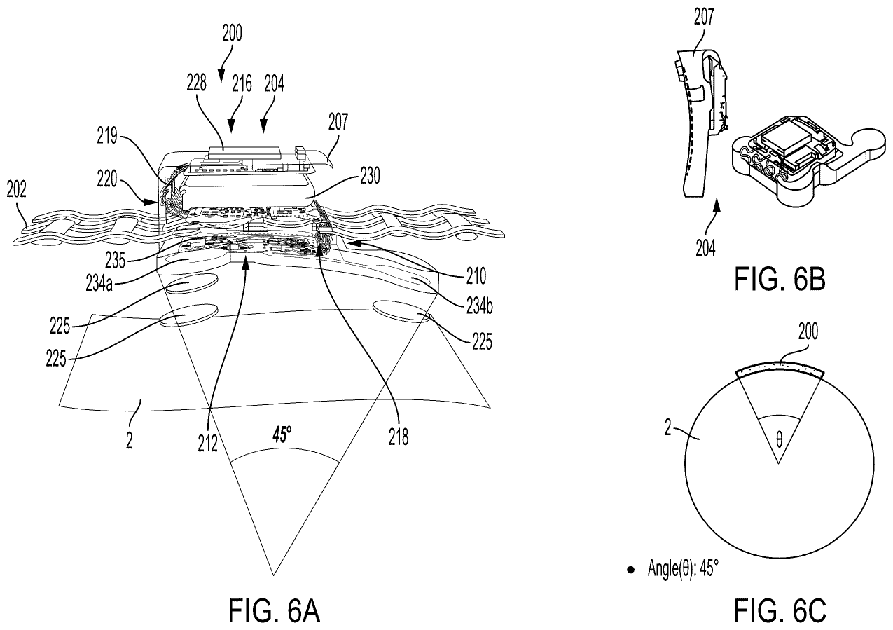

Now referring to FIGS. 5 and 6, a second embodiment of a wearable device 200 is illustrated. The wearable device 200 provides a similar structure and function as wearable device 100 and the associated components and functions provided above, except as further described herein.

The wearable device 200 has a foldable and pre-curved design as discussed further herein to enhance wearability and signal sensitivity. The wearable device 200 may have a mesh-type textile 202 to facilitate breathability and stretchability for a comfortable and appropriate fit encircling a human subject's neck 2. The textile 202 may be embedded within a sensor system 204 for proper positioning of the sensor system 204 relative to the human subject's neck 2.

The sensor system 204 may be a three-layered structure and/or foldable and/or pre-curved to about 45°. The sensor system 204 includes an interior electronics and sensor assembly 210. The interior electronics and sensor assembly 210 may include a flexible printed circuit board 235 for facilitating the flexible and/or pre-curved design of the sensor system 204. The sensor system 204 may include a number of other active and passive components, including an accelerometer 222, a microphone 223, at least one surface electromyography (sEMG) measurement electrode 224a for measuring and transmitting data related to consumption, movement, and other human activities as discussed further herein, an sEMG reference electrode 224b, and an accompanying sEMG amplifier 226. As illustrated, sensor system 204 may include two sEMG measurement electrodes 224a. The sensor system 204 may further include a microcontroller 228, a power source 230 with corresponding charging circuit, and a power converter (i.e., power management circuit with optional regulator) 234.

The accelerometer may be a three-axis accelerometer or another accelerometer for carrying out the functions as described further herein. For example, the accelerometer may be configured for comprehensive motion measurement and offer a sampling frequency and resolution of up to 1,600 Hz and 16 bits, respectively, with a broad bandwidth response (e.g., 0-1,600 Hz and a sufficient dynamic range of 2 g, where g denotes gravitational acceleration of 9.8 ms⁻²) The sEMG amplifier may, for example, feature a 10-400 Hz bandwidth and 5,000 gains. The microphone may, for example, be an ultra-low noise microelectronic mechanical system microphone with a 70 dB signal-to-noise ratio configured to detect acoustic signals from the neck area as described further herein. The microcontroller may be, for example, a Bluetooth® system-on-chip semiconductor or any other semiconductor or processor configured for data acquisition from the accelerometer, surface electromyography recording electrode and amplifier, and microphone, and wireless communication of the sensor data to a user interface. In another embodiment, another accelerometer, sEMG amplifier, microphone, and/or microcontroller may be used according to the required functions as described herein.

Power source 230 may include a lithium-ion battery, such as a 150 mAh lithium-ion polymer batter. The power source is preferably rechargeable and may provide approximately 18 hours or more of power before recharging is needed via the associated charging circuit. In some embodiments, the power source may provide 5, 10, 12, 14, 16, 18, 20, 22, 24 or more hours of power before recharging is needed. In other embodiments, the power source may provide multiple days of power before recharging is needed. The power converter is configured to convert the power held and distributed from the power source to a voltage necessary for operation of the remaining components. For example, the power converter may be configured to convert 3.7 V provided by the power source to 3.3 V required for component operation. The power converter may be associated with a power management circuit for distribution of the converted power to the components for operation thereof.

As shown in FIG. 5, wearable device 200 includes a first segment 212, a second segment 214, and a third segment 216, each of the segments 212, 214, 216 including components of electronics and sensor assembly 210. Segment 212 and segment 214 are joined with first bridge section 218, while segment 214 and segment 216 are joined with second bridge section 220. As illustrated, first segment 212 may include microphone 223, accelerometer 222, sEMG measuring electrodes 224a, sEMG reference electrode 224b, and amplifier 226 positioned thereon; second segment 214 may include power source 230, power converter 234, and the supporting charging circuit positioned thereon; and third segment 216 may include microcontroller 228 supported thereon.

As described further herein and illustrated in FIG. 6A, first bridge section 218 and second bridge section 220 are foldable so that first segment 212, second segment 214, and third segment 216 are stackable, i.e., placed in a stacked configuration, where second segment 214 is positioned on top of first segment 212 and third segment 216 is positioned on top of second segment 214. The sensor system 204 may be coated in an elastomer coating 207 (e.g., while in the stacked configuration). Referring additionally to FIGS. 6B-6C, the interior surface, i.e., the surface of first segment 212 facing user's neck 2, including elastomer coating 207, may be precurved at about 45° as described further herein. Elastomer coating 207 is preferably soft in texture and waterproof to offer resistance to sweat while also providing enhanced skin comfort.

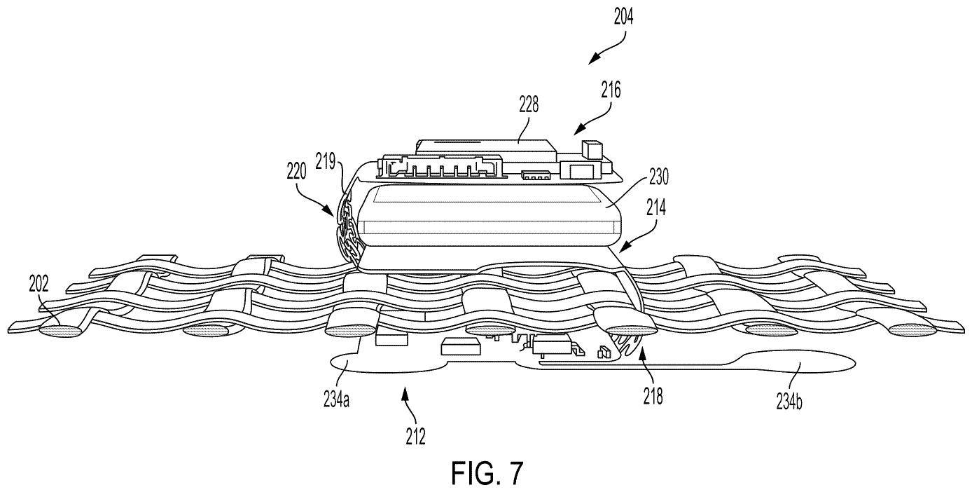

Referring to FIGS. 6A and 7, textile 202 may be positioned between first segment 212 and second segment 214 when sensor system 204 is in the stacked configuration. In some embodiments, textile 202 may be positioned prior to coating of sensor system 204 in elastomer coating 207 so as to embed textile 202 within sensor system 204. Textile 202 may be a mesh material or another type of flexible material configured to conform securely and comfortably to a user's neck. Textile 202 may include a fastening mechanism, such as a hook and loop strap, a hook and eye coupling mechanism, a buckle, or another fastening mechanism to facilitate fastening of textile 202 and attached sensor system 204 to the user's neck. In some embodiments, the fastening mechanism is preferably adjustable, allowing wearable device 202 to be one-size-fits-most or one-size-fits-all while still providing a secure and comfortable fit for the user.

Textile 202 may be configured to fit neck circumferences from about 29 cm to about 54 cm with a minimal stretch of less than 15%. In other embodiments, textile 202 may be configured to fit smaller or greater neck circumferences than the range specified above. As discussed further herein, textile 202, and therefore wearable device 200, may be configured to stretch and twist freely to facilitate a secure and comfortable fit for the user. Textile 202 may come in various shapes, colors, and materials, including, for example, acrylics, rayons, and polyesters. Mesh textiles may have a varying mesh sizes.

Referring again to FIGS. 5-7, each segment 212, 214, 216 may be similarly sized and shaped to facilitate arrangement of segments 212, 214, 216 into the stacked configuration and placement of sensor system 204 over the thyrohyoid muscle of user's neck 2 or other placement suitable for the methods disclosed herein. Each segment 212, 214, 216 may be generally square in shape. In other embodiments, each segment 212, 214, 216 may be circular, rectangular, or another shape facilitating desired placement of sensor system 204. Each segment 212, 214, 216 may be about 15 mm×15 mm, 20 mm×20 mm, or 25 mm×25 mm, for example. In other embodiments, segments 212, 214, 216 may be otherwise sized to facilitate desired placement of sensor system 204. As illustrated in FIG. 6, conductive hydrogel may be positioned on user's neck 2 corresponding with sEMG electrodes 224a, 224b to facilitate measurement of data as discussed further herein.

As discussed above, each bridge section 218, 220 may include at least one interconnector 219 for coupling of segment 212 with segment 214 and coupling of segment 214 with segment 216, respectively. In the illustrated embodiment, bridge sections 218, 220 includes a plurality of interconnections 219. In some embodiments, bridge sections 218, 220 may include four interconnections 219, as illustrated. In other embodiments, bridge sections 218, 220 may include one, two, three, five, six, seven, eight, nine, ten, or more interconnections. As illustrated, interconnections 219 may be serpentine in configuration, with a width of about 150 μm, about 175 μm, about 200 μm, about 225 μm, or about 250 μm, and where each curved portion of the serpentine configuration has an arc angle of about 250°, about 260°, about 270°, about 280°, about 290°, or about 300°. Other interconnection configurations may be utilized which facilitate the movement capability of wearable device 200 and function thereof as described further herein.

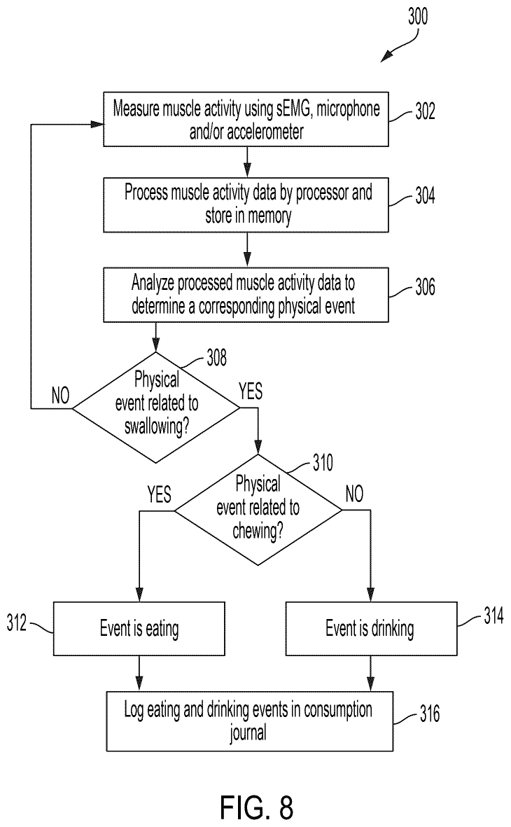

Now referring to FIG. 8, a method 300 of using wearable device 100, 200 (FIGS. 1, 5) is illustrated. When wearable device 100, 200 is worn and positioned as described above, sEMG recording electrodes 124, 224, microphone 123, 223, and accelerometer 122, 222 may be positioned to measure activity of an underlying muscle as shown at box 302. In aspects, muscle activity may be measured over a recurring sampling period, such as 100 milliseconds, 500 milliseconds, on) second, two (2) seconds, five (5) seconds, or any other suitable recurring sampling period; in other aspects, muscle activity may be measured over a periodic sampling period. For instance, a periodic sampling period may be triggered by a detection of muscle activity whereupon the sEMG may begin recording measurements. Microcontroller 128, 228 may process the muscle activity data, including the date, the time, or the date and time at which the data was collected, which may then be stored within memory 132, 232 for further processing as shown at box 304. In aspects, further processing may include, for example, converting sensor data from the sEMG, microphone, and/or the accelerometer into data representative of muscle activity in volts (V) and/or hertz (Hz), respectively. In some aspects, the muscle activity data may be processed in chunks, e.g., by aggregating the muscle activity data over a period of time and processing the aggregated data as a chunk. Memory 132, 232 may be accessed, either by removing memory 132, 232 from wearable device 100, 200 as described above to be read by another memory-readable device (e.g., computing device 148) or by creating a wired or wireless connection between memory 132, 232 and another memory-readable device (e.g., computing device 148 as described above).

At box 306, the processed muscle activity data (processed data) may be further analyzed to identify at least one corresponding physical event consistent with the measured muscle activity data from box 302. This analysis may be performed, for example, using a machine-learning algorithm that is trained to associate data patterns corresponding to physical events for automatically identifying physical events corresponding to the muscle activity data, including distinguishing the identified physical events between food-consumption and non-food-consumption events. The aforementioned machine-learning algorithm may be implemented wholly within microcontroller 128, 228 wholly within processing unit 158 of remote computing device 148, or cooperatively between both processors such that both processors work together to implement the machine-learning algorithm. In some aspects, other methods may be used to identify corresponding physical events based on the processed data, for example, by comparing newly processed data to previously processed data corresponding to identified physical events. The identified physical events may include a variety of food-consumption or non-food-consumption events, including, for example, body orientation, walking, running, talking, sitting down, head and jaw movement, and other events that may require or use movement or other activity of the muscles underlying wearable device 100, 200.

In some embodiments, if the identified physical events are determined to be non-food-consumption events, such as lying down with lack of further activity, walking with lack of further activity, running with lack of further activity, sitting down with lack of further activity, and speaking with lack of further activity, such physical events are treated as a false condition and dismissed at box 306. Other physical events which may be related to food consumption, such as opening and closing of the mouth, chewing, or swallowing, may be further analyzed at box 308. In some embodiments, all identified physical events, regardless of whether they are determined to be related to food consumption or not, are further analyzed at box 208. In aspects, at box 308, when a food-consumption event is associated with swallowing, the method may progress to box 310. Alternatively, at box 308, when a food-consumption related event is not associated with swallowing, the method may return to box 302. Examples of food-consumption events that do not involve swallowing, for example, may include opening and/or closing of the mouth to talk.

At box 310, when the food-consumption event is associated with swallowing, the processed data may be analyzed further to determine whether the physical event is related to chewing. If so, the food-consumption event is associated with both swallowing (at box 308) and chewing (at box 310). In this case, at box 312, it may be determined that the food-consumption event is eating. If not, when the food-consumption event is related to swallowing (at box 308) but not chewing (at box 310), it may be determined that the food-consumption event is drinking at box 314.

In further aspects, eating or drinking food-consumption events may be logged to create a consumption journal at box 316. The consumption journal may include the processed data related to the food and/or drink event, along with the time and date such data was collected. Such consumption journal may then be used by a provider to, for example, instruct a human subject to adjust food, liquids, or medication for food-related illnesses, such as obesity or diabetes.

### Methods

### Fabrication of Tested Wearable Device

The wearable device as described throughout the examples below is generally consistent with at least one or both of wearable devices 100, 200 as described above. In preparation for at least Examples 20-23 described below, a wearable device was prepared as described further herein. The demonstrations provided by said examples are applicable to either or both of wearable device 100, 200.

Commercial software was used to generate schematic diagrams and layouts for the tested flexible printed circuit board. The wearable device was designed with three segments in a stacked configuration as discussed above, incorporating commercially available electronic components. The first segment housed a three-axis digital accelerometer, a custom-designed sEMG amplifier, and a microphone. Battery charging and voltage regulator circuits were located in the second segment, while the third segment housed a Bluetooth® Low Energy system-on-chip microcontroller and LEDs. The thyrohyoid sEMG signal measurements employed small diameter electrodes (6 mm) having a small interelectrode distance (20 mm) to reduce detection volume and minimize crosstalk effects, consistent with the surface electromyography for the non-invasive assessment of muscles (Surface ElectroMyoGraphy for the Non-Invasive Assessment of Muscles, or SENIAM) guidelines. Customized firmware was uploaded to the microcontroller. After the various surface-mount components were placed, they were soldered to the flexible printed circuit board, and the system was folded, marking the completion of the electronics' fabrication. Solder paste was used to attach the various surface-mount components to the flexible printed circuit board, facilitated by a heat gun and hot plate. Once soldering was completed, the flexible printed circuit board was covered with a silicon conformal coating to strengthen the solder bonding. An off-the-shelf mesh textile neckband was then placed between the first and second layers of the flexible printed circuit board to integrate the neckband. The entire structure was subsequently encapsulated with a soft, waterproof elastomer to shield the system from external factors and offer a sturdy, long-lasting interface for manipulation. To guarantee a secure attachment of the neckband to the skin, the skin interface layer was made of a conductive hydrogel for the sEMG electrodes. This layer was shaped using a 6 mm round hole punch to match the electrode layout of the wearable device.

A mold was designed using commercial 3D CAD software and was printed using a stereolithography 3D printer. Before starting the encapsulation process, a silicone release agent was applied. The encapsulation was carried out with the fully fabricated sensor module and a textile neckband positioned within the mold. A soft elastomer gel was utilized as the encapsulation layer.

### Methods for Analysis

The SNR as disclosed and discussed further herein was compared using power spectral density estimation in MATLAB. The SNR in decibels (dB) was calculated using the following formula:

$${SNR}_{dB} = 10 {log}_{10} ( \frac{{P}_{signal}}{{P}_{noise}} )$$

### EXAMPLES

### Example 1

A wearable device having an electronics and sensor assembly generally consistent with the electronics and sensor assembly 110 discussed above was worn through three consecutive swallowing events. The sensor assembly included sEMG recording electrodes consistent with sEMG recording electrodes 124a, 124b, 124c described above and a signal amplifier circuit consistent with the signal amplifier circuit 126 described above. The resultant activity measured by the sEMG recording electrodes was measured and recorded.

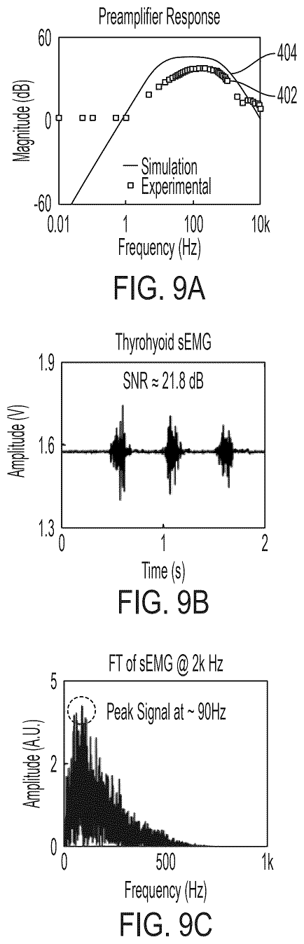

FIG. 9A illustrates the response of the signal amplifier circuit in terms of magnitude and frequency, wherein line 302 illustrates the raw experimental data and line 404 illustrates the simulated curve in view of said experimental data. As shown, the required range of the signal amplifier circuit is from about 10 Hz to about 500 Hz. FIG. 9B illustrates a thyrohyoid muscle sEMG sampled at 2 kHz during the three consecutive swallows, which demonstrates a signal-to-noise ratio of 21.8 dB. FIG. 9C illustrates a Fourier transform of the same sEMG signal, showing the peak power is located around 90 Hz, demonstrating that the sampling rate may be reduced from 1 kHz to about 200 Hz to reduce power consumption and increase battery life of the electronics and sensor assembly.

### Example 2

A wearable device having an electronics and sensor assembly generally consistent with the electronics and sensor assembly 110 discussed above was worn through two consecutive swallowing events at varying sEMG sampling rates, the results of which were measured and recorded.

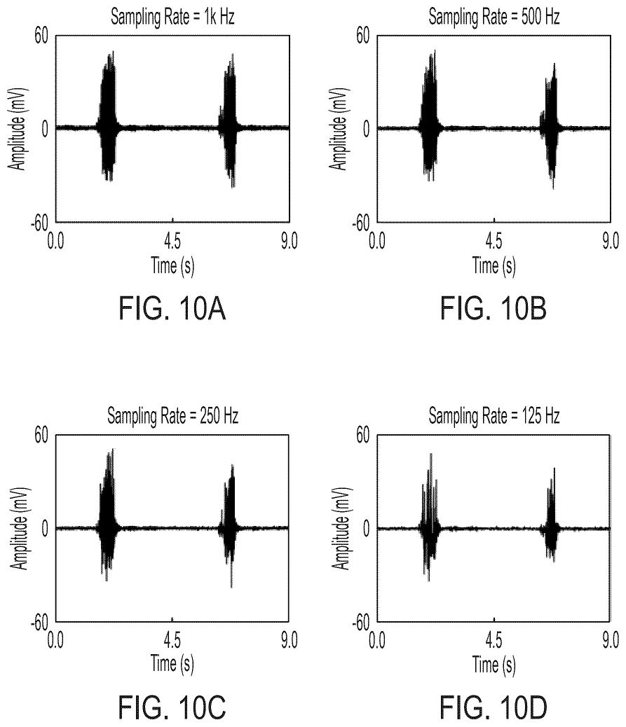

FIG. 10A illustrates the measured amplitude of the swallowing events at a sampling rate of 1 kHz. FIG. 10B illustrates the measured amplitude of the swallowing events at a sampling rate of 500 Hz. FIG. 10C illustrates the measured amplitude of the swallowing events at a sampling rate of 250 Hz. FIG. 10D illustrates the measured amplitude of the swallowing events at a sampling rate of 125 Hz. As shown, each swallowing event produces a significant amplitude measurement at each of the tested sampling rates, and such amplitude is generally consistent at 250 Hz, 500 Hz, and 1 kHz. As such, the sampling rate for the electronics and sensor assembly may be reduced from a conventionally accepted rate of 1 kHz based on the Nyquist criterion to 250 Hz to reduce power consumption and increase operation time without sacrificing signal fidelity to detect thyrohyoid muscle activity for the activity monitoring as discussed above.

### Example 3

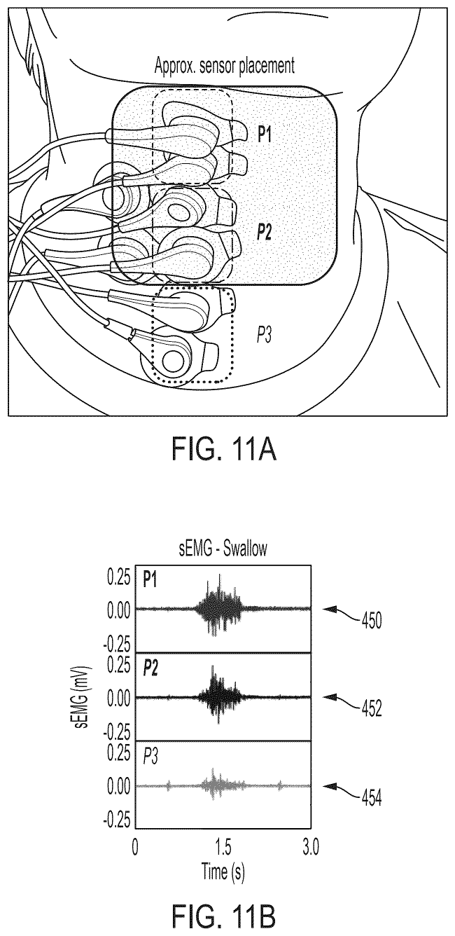

sEMG recording electrodes generally consistent with the sEMG recording electrodes 124a, 124b, 124c described above were positioned as shown in FIG. 11A, with a positive and negative sEMG recording electrode pair positioned at “P1” corresponding to the thyrohyoid area, a positive and negative sEMG recording electrode pair positioned at “P2” corresponding to the laryngeal prominence (i.e., the Adam's apple), and a positive and negative sEMG recording electrode pair positioned at “P3” corresponding to the suprasternal notch area. The amplitude of the sEMG signal was then measured and recorded during a swallowing event.

As shown in FIG. 11B, amplitude 450 illustrates the amplitude of the sEMG recording electrodes at position “P1”. Amplitude 452 illustrates the amplitude of the sEMG recording electrodes at position “P2”. Amplitude 454 illustrates the amplitude of the sEMG recording electrodes at position “P3”. As demonstrated, the measured amplitude decreases as the sEMG recording electrodes are placed further away from the thyrohyoid area. In other words, amplitude 450 is greater than amplitude 452, which is greater than amplitude 454.

### Example 4

A wearable device having an electronics and sensor assembly generally consistent with the electronics and sensor assembly 110 discussed above was worn through a number of physical events while the accelerometer and sEMG recording electrodes measured the underlying muscle activity throughout each physical event. The physical events included remaining stationary for 24 seconds, walking for 54 seconds, speaking for 25 seconds, and eating for 36 seconds.

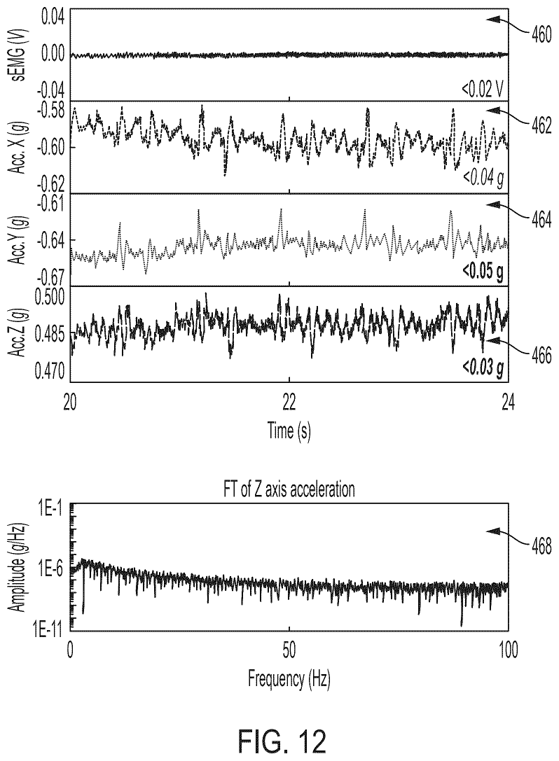

FIG. 12 illustrates the measured muscle activity over seconds 20-24 of a stationary period. The measured thyrohyoid sEMG activity is provided at graph 460, the measured X-axis acceleration is provided at graph 462, the measured Y-axis acceleration is provided at graph 464, the measured Z-axis acceleration is provided at graph 466, and a Fourier transform of the Z-axis acceleration represented in the frequency domain is provided at graph 468. As shown by graph 460, the thyrohyoid sEMG shows no detected muscle activity over the 24 second stationary period. The acceleration measurements shown by graphs 462, 464, and 466 show that over the 24 second stationary period, the measured acceleration is less than 0.05 g. The sloping baseline is reflective of subtle body movement, while the peaks shown in graphs 462, 464, and 466 are generally consistent with a pulse corresponding to the wearer's heartbeat, which presents as a mechanical movement on the skin surface near the throat area.

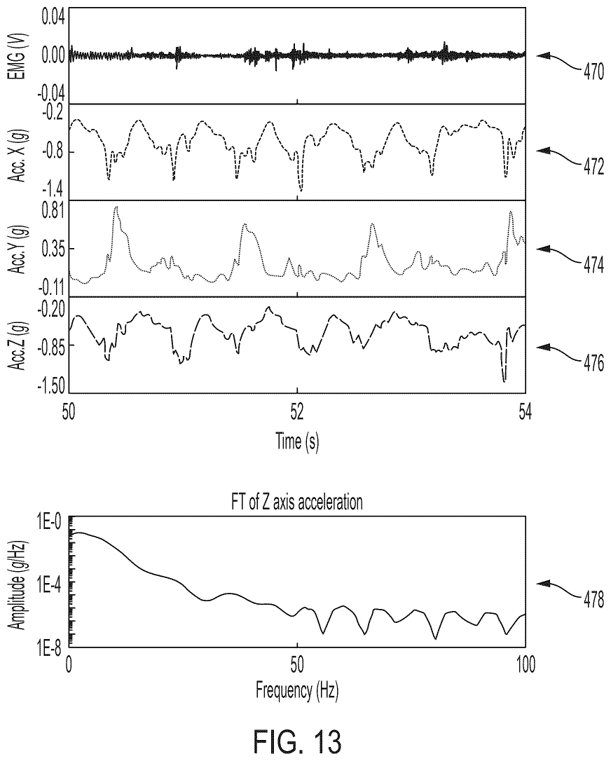

FIG. 13 illustrates the measured muscle activity over seconds 50-54 of a walking period. The thyrohyoid sEMG activity is provided at graph 470, the measured X-axis acceleration is provided at graph 372, the measured Y-axis acceleration is provided at graph 474, the measured Z-axis acceleration is provided at graph 476, and a Fourier transform of the Z-axis acceleration represented in the frequency domain is provided at graph 478. The motion artifact shown in graph 470 is indicative of a large physical movement, while the acceleration measurements shown in graphs 472, 474, and 476 reflects a large signal variation of about 1 g, reflecting movement of the body during the walking event. Each signal peak in graph 474 corresponds to two peaks in graphs 472 and 476. This is because the acceleration measurement taken along the Y-axis and illustrated in graph 474 is representative of the set of left and right steps taken, while the acceleration measurements taken along each of the X-axis and the Z-axis are representative of each step taken. The Fourier transform of the Z-axis acceleration illustrated in graph 478 shows peak amplitude at a low-frequency region of less than 5 Hz, which further reflects the large and low-frequency signals in graphs 470, 472, 474, and 476.

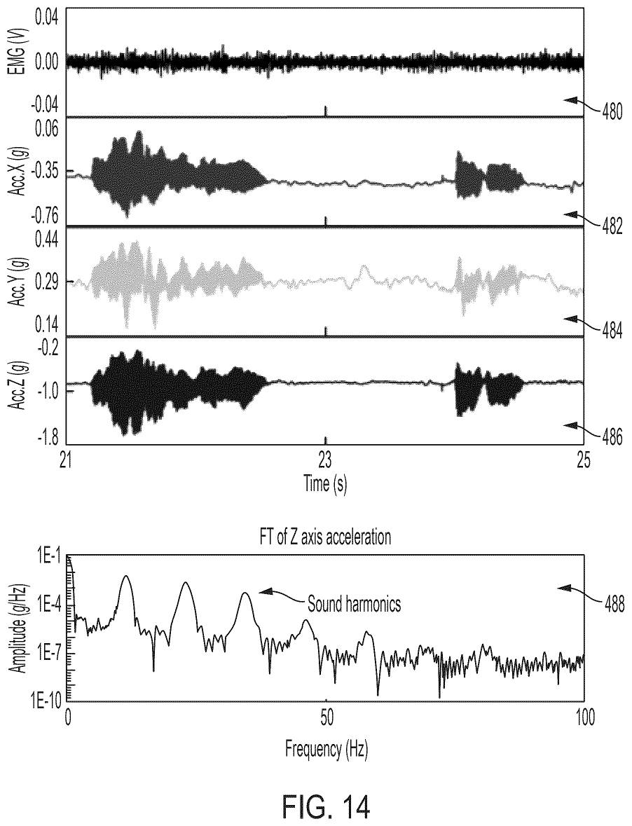

FIG. 14 illustrates the measured muscle activity over seconds 21-25 of a talking period. The thyrohyoid sEMG activity is provided at graph 480, the measured X-axis acceleration is provided at graph 482, the measured Y-axis acceleration is provided at graph 484, the measured Z-axis acceleration is provided at graph 486, and a Fourier transform of the Z-axis acceleration represented in the frequency domain is provided at graph 488. The thick baseline without motion artifact indicates the lack of a large body movement during the speaking event. In contrast, the acceleration data shows a high-frequency signal each of graphs 482, 484, and 486 corresponding with each of the X-axis, the Y-axis, and the Z-axis, respectively, during the vocal activity. The vocal activity can also be seen in the corresponding Fourier transform in graph 488 as repeated peaks (sound harmonics).

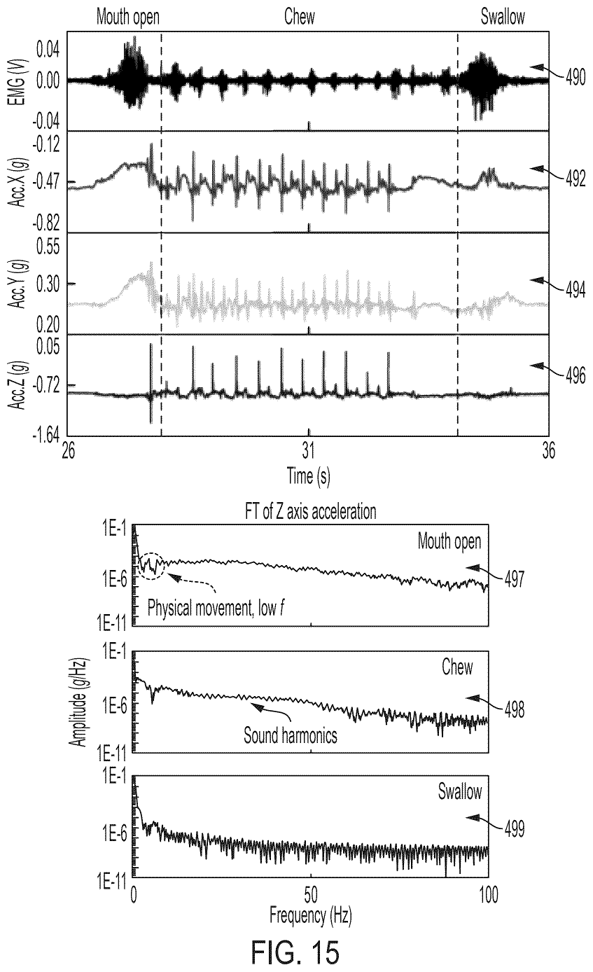

FIG. 15 illustrates the measured muscle activity over seconds 26-36 of an eating event, including subevents of a wearer's mouth opening, chewing, and swallowing. The thyrohyoid sEMG activity is provided at graph 490, the measured X-axis acceleration is provided at graph 492, the measured Y-axis acceleration is provided at graph 494, the measured Z-axis acceleration is provided at graph 496, a Fourier transform of the Z-axis acceleration represented in the frequency domain during the physical mouth opening subevent is provided at graph 497, a Fourier transform of the Z-axis acceleration representing sound harmonics in the frequency domain during the chewing subevent is provided at graph 498, and a Fourier transform of the Z-axis acceleration represented in the frequency domain during the swallowing subevent is provided at graph 499. As illustrated by graph 490, distinct thyrohyoid muscle activity can be measured for each jaw movement corresponding with the mouth opening and chewing subevents as well as the swallowing subevent. Acceleration data as illustrated in graphs 492, 494, and 496 also demonstrates a distinct signal pattern along each of the X-axis, the Y-axis, and the Z-axis, respectively, for each of the movements, which are also illustrated in the Fourier transform as a distinctive signal pattern in graphs 497, 498, and 499.

### Example 5

A wearable device having an electronics and sensor assembly generally consistent with the electronics and sensor assembly 110 discussed above was worn through a number of physical events while the accelerometer and sEMG recording electrodes measured the underlying muscle activity throughout each physical event. A microphone was further used to record sound pressure waves throughout each of the physical events. The physical events included remaining stationary for 18 seconds, talking while sitting for 18 seconds, walking at a speed of about 3.5 km/h for 18 seconds, chewing and eating solid food in the form of sweet potato chips for 55 seconds, and drinking water for 55 seconds. In aspects, the 2D and 3D spectrograms described below illustrate different frequency patterns for different physical events, such as being stationary, talking, walking, chewing, and swallowing. For example, the acceleration represented by frequency patterns associated with the X, Y, and Z axes can be used to eliminate “talking” related signals from the swallowing and chewing signals. In addition, when distinguishing between swallowing and chewing, the low-frequency band is dominant in chewing as compared to swallowing. In this way, spectrogram frequency patterns may be used to distinguish between talking, chewing, and swallowing signals.

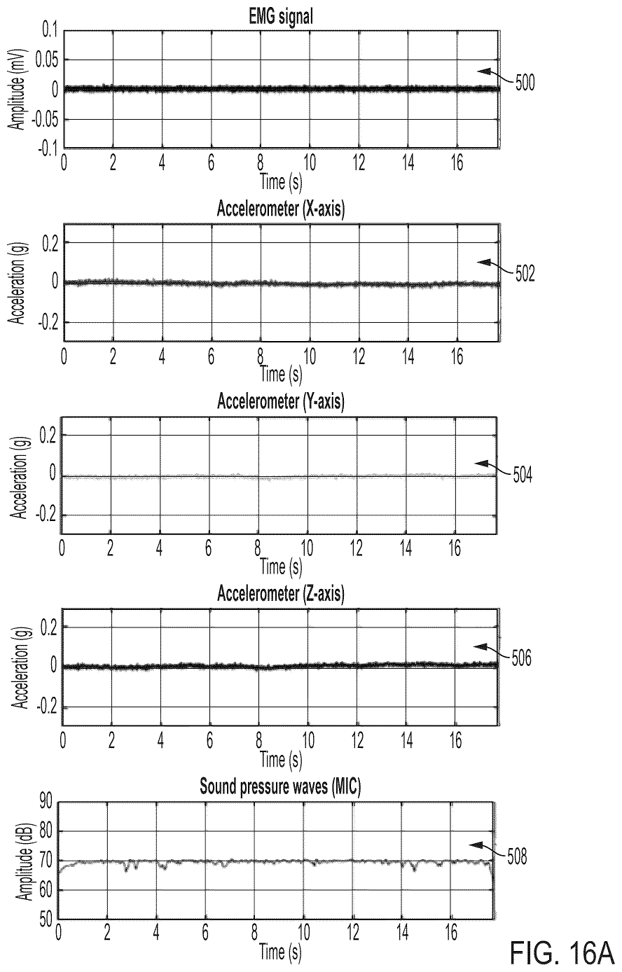

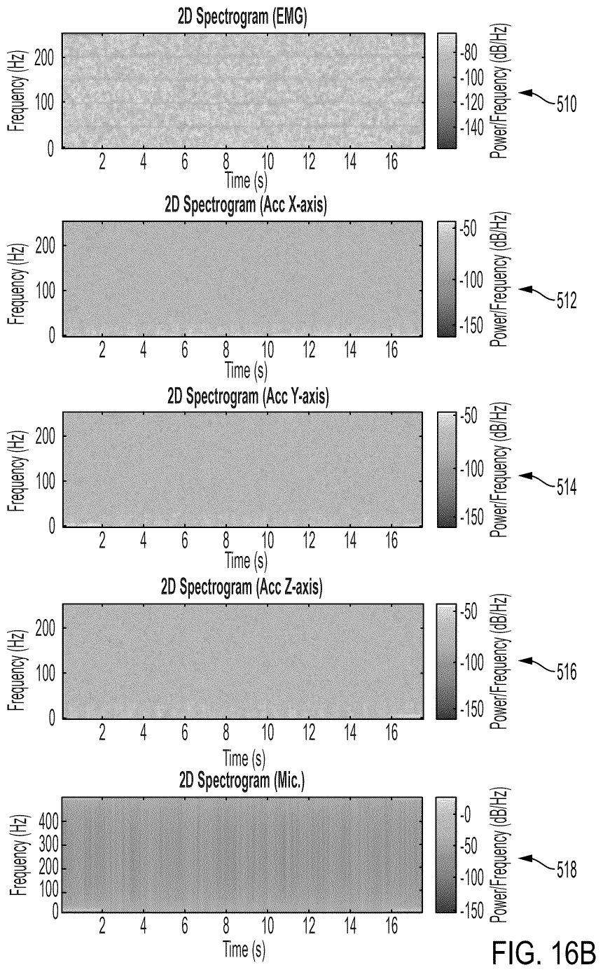

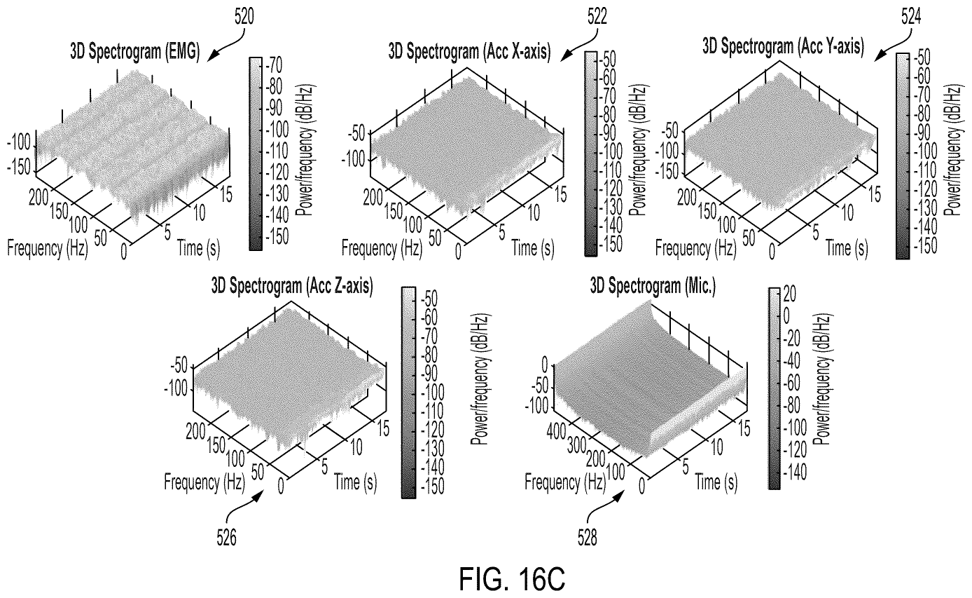

FIGS. 16A-16C illustrate the measured muscle activity and sound pressure waves over 18 seconds of a seated stationary event. The thyrohyoid sEMG activity is provided at graph 500 (FIG. 16A), with a corresponding two-dimensional spectrogram provided at graph 510 (FIG. 16B) and a corresponding three-dimensional spectrogram provided at graph 520 (FIG. 16C). The measured X-axis acceleration is provided at graph 502 (FIG. 16A), with a corresponding two-dimensional spectrogram provided at graph 512 (FIG. 16B) and a corresponding three-dimensional spectrogram provided at graph 522 (FIG. 16C). The measured Y-axis acceleration is provided at graph 504 (FIG. 16A), with a corresponding two-dimensional spectrogram provided at graph 514 (FIG. 16B) and a corresponding three-dimensional spectrogram provided at graph 524 (FIG. 16C). The measured Z-axis acceleration is provided at graph 506 (FIG. 16A), with a corresponding two-dimensional spectrogram provided at graph 516 (FIG. 16B) and a corresponding three-dimensional spectrogram provided at graph 526 (FIG. 16C). The measured sound pressure waves are provided at graph 508 (FIG. 16A), with a corresponding two-dimensional spectrogram provided at graph 518 (FIG. 16B) and a corresponding three-dimensional spectrogram provided at graph 528 (FIG. 16C).

As shown in graph 500, the sEMG signal remains within a 0.05 mV amplitude throughout the stationary event. The measured accelerations along each of the X-axis, the Y-axis, and the Z-axis illustrated in each of graphs 502, 504, and 506, respectively, remain within a 0.2 g acceleration throughout the stationary event. The sound pressure waves illustrated in graph 508 similarly remains generally consistent throughout the stationary period. The changes in amplitude illustrated within graph 508 may be attributed to the wearer's heartbeat, as the electronics and sensor assembly is positioned near the wearer's pulse point.

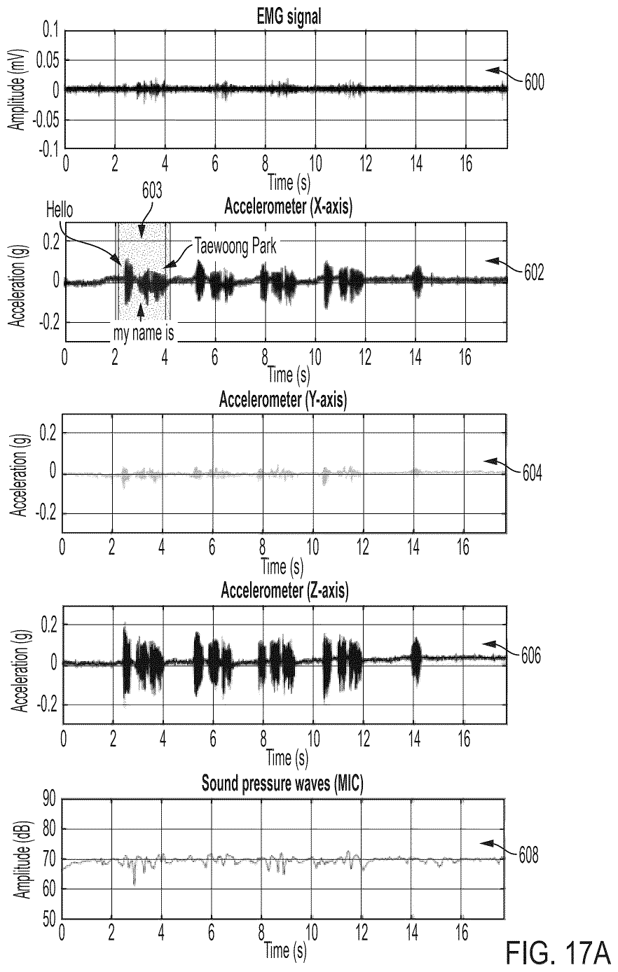

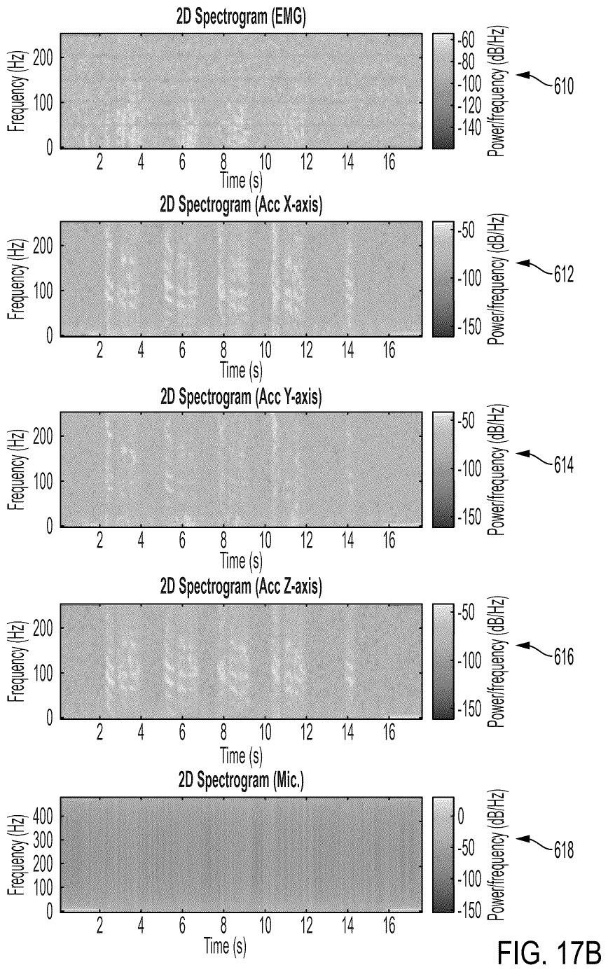

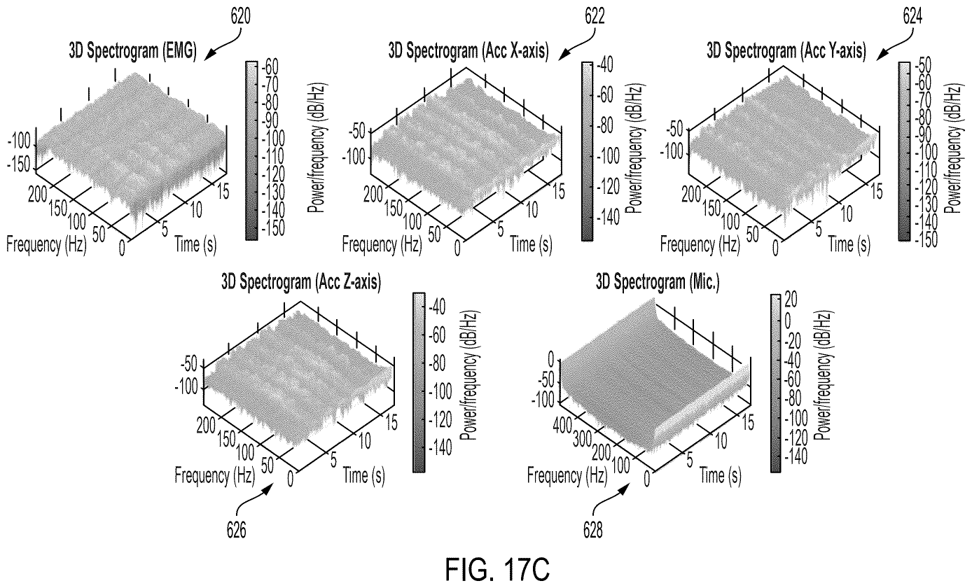

FIGS. 17A-17C illustrate the measured muscle activity and sound pressure waves over 18 seconds of a speaking event. The thyrohyoid sEMG activity is provided at graph 600 (FIG. 17A), with a corresponding two-dimensional spectrogram provided at graph 610 (FIG. 17B) and a corresponding three-dimensional spectrogram provided at graph 620 (FIG. 17C). The measured X-axis acceleration is provided at graph 602 (FIG. 17A), with a corresponding two-dimensional spectrogram provided at graph 612 (FIG. 17B) and a corresponding three-dimensional spectrogram provided at graph 622 (FIG. 17C). The measured Y-axis acceleration is provided at graph 604 (FIG. 17A), with a corresponding two-dimensional spectrogram provided at graph 614 (FIG. 17B) and a corresponding three-dimensional spectrogram provided at graph 624 (FIG. 17C). The measured Z-axis acceleration is provided at graph 606 (FIG. 17A), with a corresponding two-dimensional spectrogram provided at graph 616 (FIG. 17B) and a corresponding three-dimensional spectrogram provided at graph 626 (FIG. 17C). The measured sound pressure waves are provided at graph 608 (FIG. 17A), with a corresponding two-dimensional spectrogram provided at graph 618 (FIG. 17B) and a corresponding three-dimensional spectrogram provided at graph 628 (FIG. 17C).

As shown in graph 600, the sEMG signal includes some amplitude variance while speaking. The measured accelerations along each of the X-axis, the Y-axis, and the Z-axis illustrated in each of graphs 602, 604, and 606, respectively, shows a spike in frequency during the vocal events, which are further highlighted at point 603. The sound pressure waves illustrated in graph 608 predictably change throughout the speaking event.

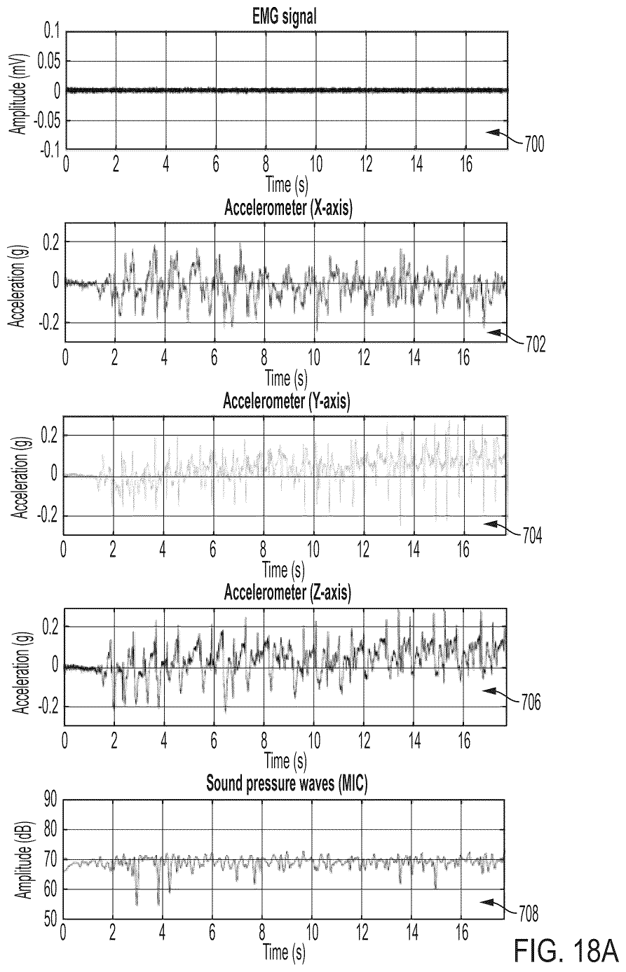

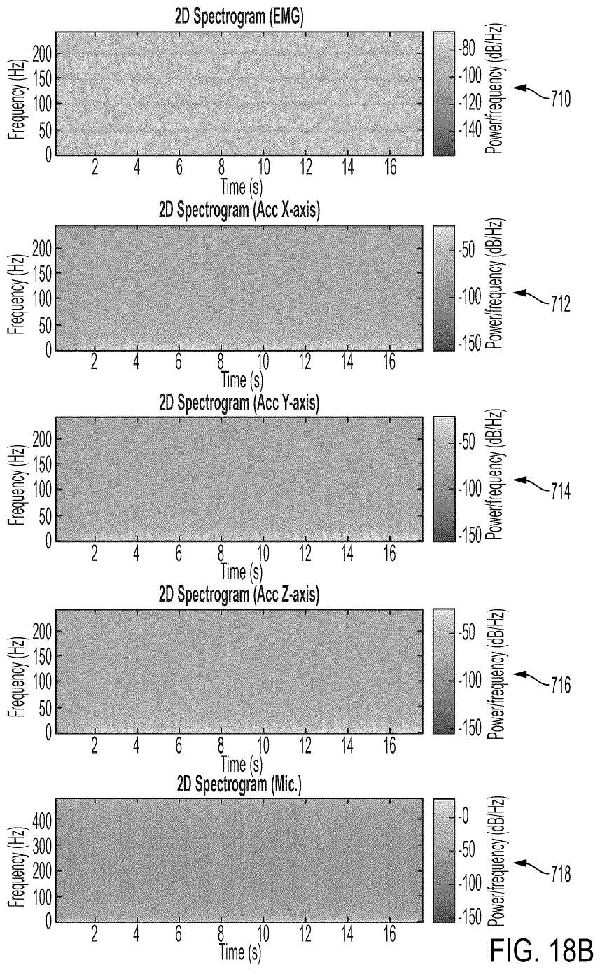

FIGS. 18A-18C illustrate the measured muscle activity and sound pressure waves over 18 seconds of a walking event at 3.5 km/h. The thyrohyoid sEMG activity is provided at graph 700 (FIG. 18A), with a corresponding two-dimensional spectrogram provided at graph 710 (FIG. 18B) and a corresponding three-dimensional spectrogram provided at graph 720 (FIG. 18C). The measured X-axis acceleration is provided at graph 702 (FIG. 18A), with a corresponding two-dimensional spectrogram provided at graph 712 (FIG. 18B) and a corresponding three-dimensional spectrogram provided at graph 722 (FIG. 18C). The measured Y-axis acceleration is provided at graph 704 (FIG. 18A), with a corresponding two-dimensional spectrogram provided at graph 714 (FIG. 18B) and a corresponding three-dimensional spectrogram provided at graph 724 (FIG. 18C). The measured Z-axis acceleration is provided at graph 706 (FIG. 18A), with a corresponding two-dimensional spectrogram provided at graph 716 (FIG. 18B) and a corresponding three-dimensional spectrogram provided at graph 726 (FIG. 18C). The measured sound pressure waves are provided at graph 708 (FIG. 18A), with a corresponding two-dimensional spectrogram provided at graph 718 (FIG. 18B) and a corresponding three-dimensional spectrogram provided at graph 728 (FIG. 18C).

As shown in graph 700, the sEMG signal remains at a baseline similar to that measured during the stationary event. However, the measured accelerations along each of the X-axis, the Y-axis, and the Z-axis illustrated in each of the graphs 702, 704, and 706, respectively, show acceleration for each movement during the walking event. Similarly, the measured sound pressure waves illustrated in graph 708 experience change throughout the walking event.

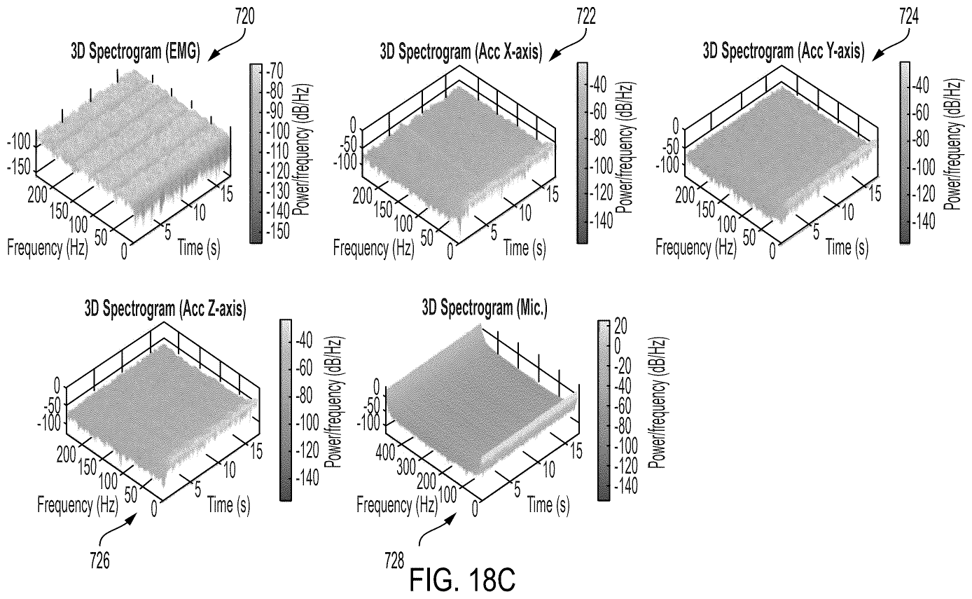

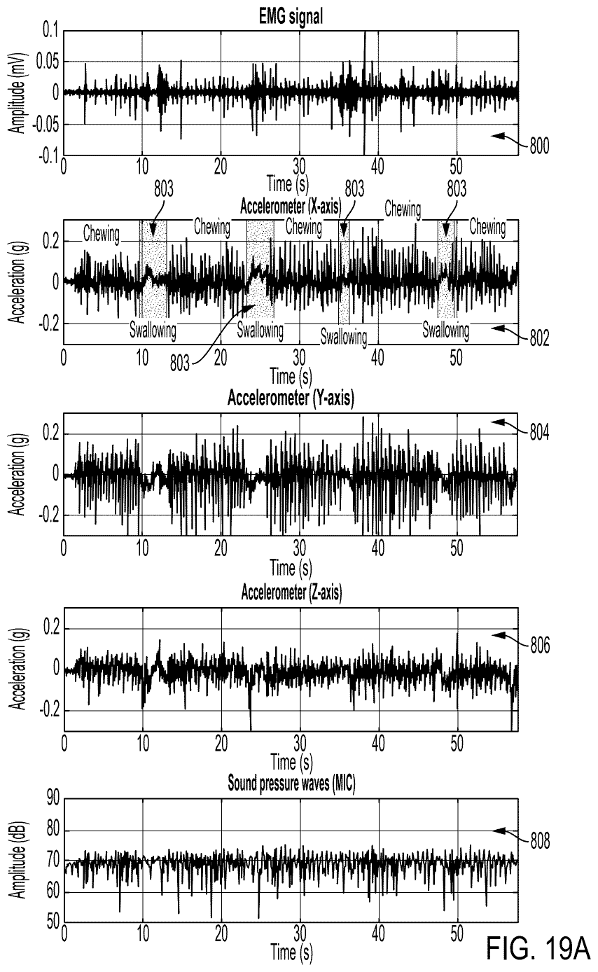

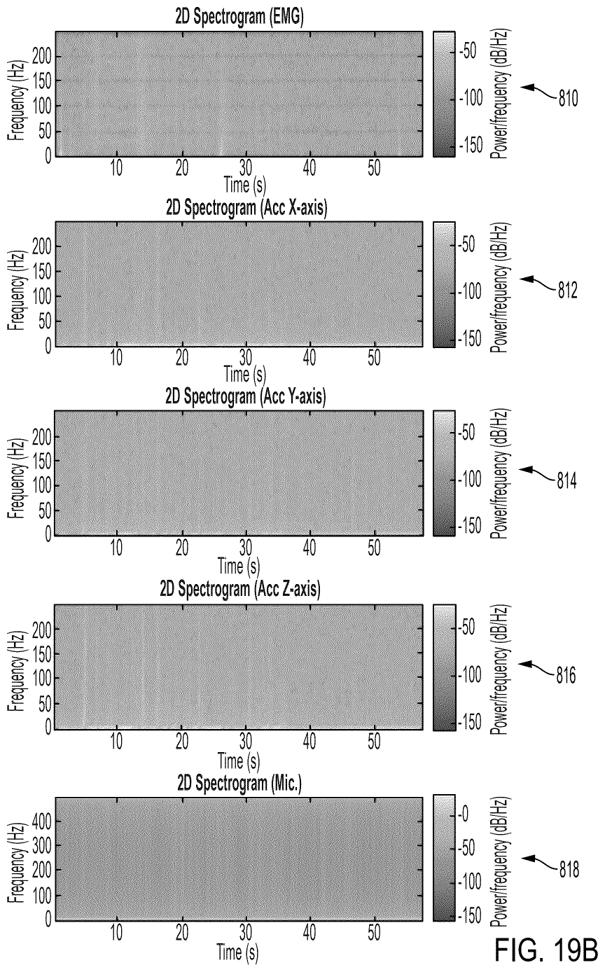

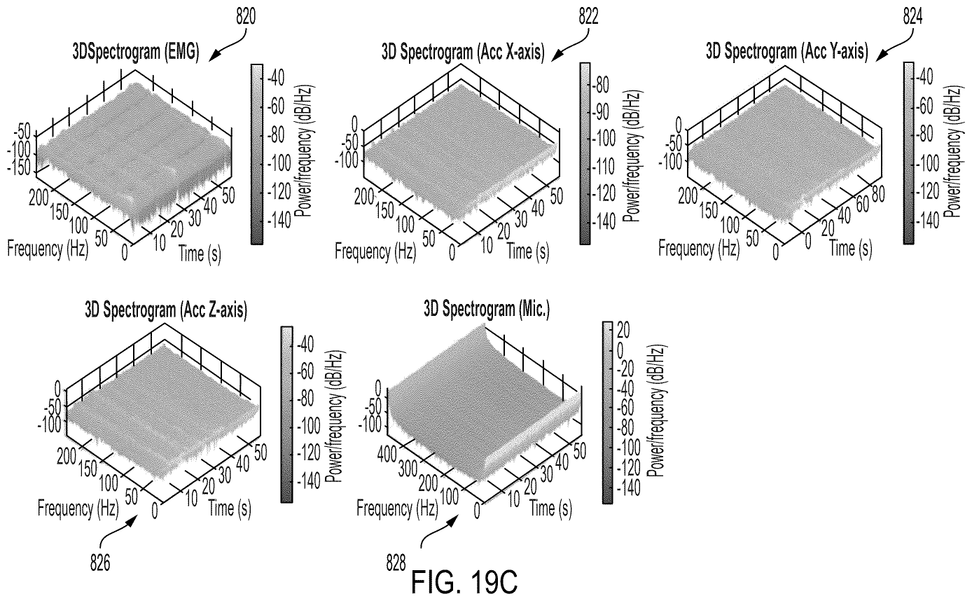

FIGS. 19A-19C illustrate the measured muscle activity and sound pressure waves over a 55 second eating event including chewing and swallowing of solid food. The thyrohyoid sEMG activity is provided at graph 800 (FIG. 19A), with a corresponding two-dimensional spectrogram provided at graph 810 (FIG. 19B) and a corresponding three-dimensional spectrogram provided at graph 820 (FIG. 19C). The measured X-axis acceleration is provided at graph 802 (FIG. 19A), with a corresponding two-dimensional spectrogram provided at graph 812 (FIG. 19B) and a corresponding three-dimensional spectrogram provided at graph 822 (FIG. 19C). The measured Y-axis acceleration is provided at graph 804 (FIG. 19A), with a corresponding two-dimensional spectrogram provided at graph 814 (FIG. 19B) and a corresponding three-dimensional spectrogram provided at graph 824 (FIG. 19C). The measured Z-axis acceleration is provided at graph 806 (FIG. 19A), with a corresponding two-dimensional spectrogram provided at graph 816 (FIG. 19B) and a corresponding three-dimensional spectrogram provided at graph 826 (FIG. 19C). The measured sound pressure waves are provided at graph 808 (FIG. 19A), with a corresponding two-dimensional spectrogram provided at graph 818 (FIG. 19B) and a corresponding three-dimensional spectrogram provided at graph 828 (FIG. 19C).

As shown in graph 800, the sEMG signal varies in a distinct manner when the wearer is chewing versus when the wearer is swallowing (i.e., compare swallowing points 803 of graph 802 with the corresponding time frames of graph 800). The same pattern can be found in the measured accelerations along each of the X-axis, the Y-axis, and the Z-axis illustrated in each of the graphs 802, 804, and 806, respectively, when the swallowing points 803 of graph 802 are compared with the corresponding time frames of graphs 804 and 806. The measured sound pressure waves illustrated in graph 808 also experience change throughout the eating event.

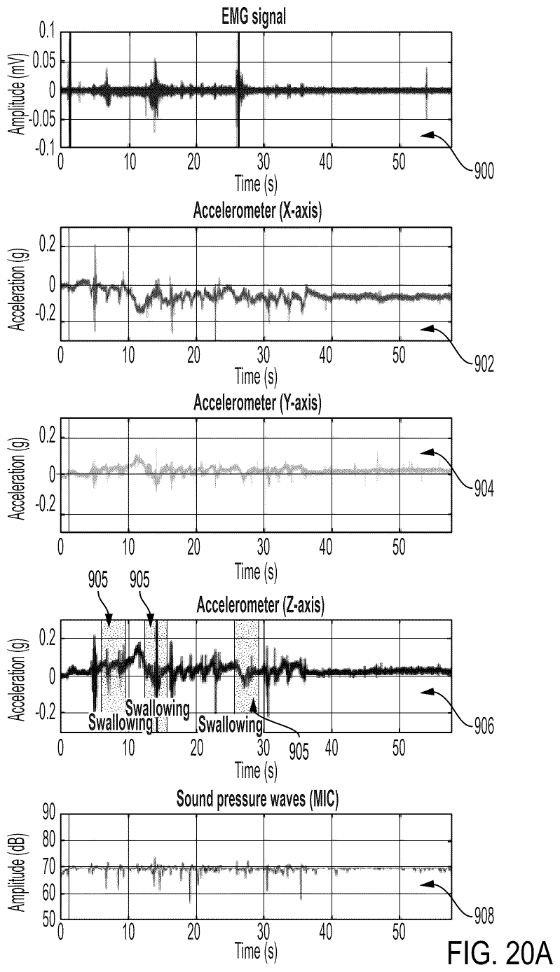

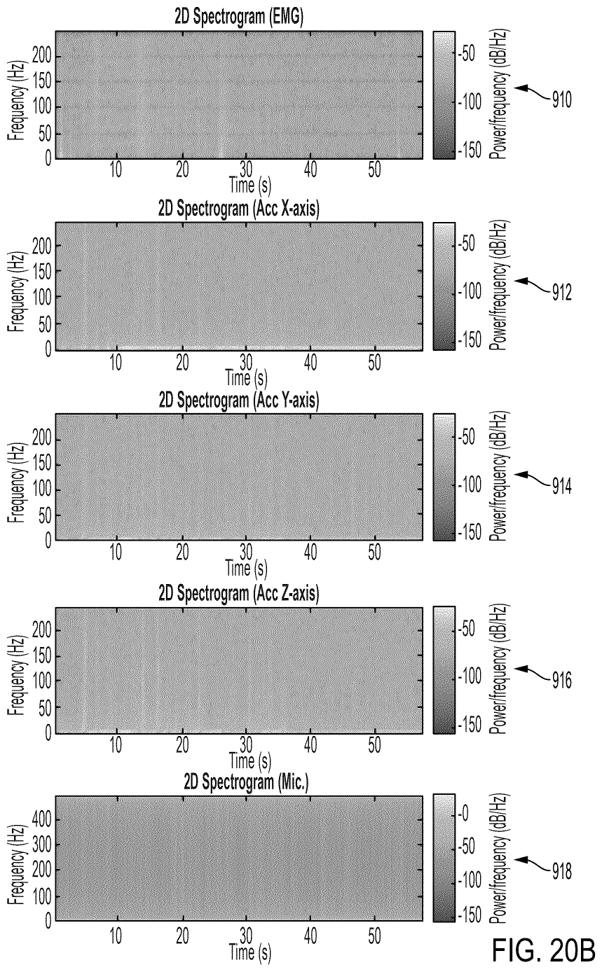

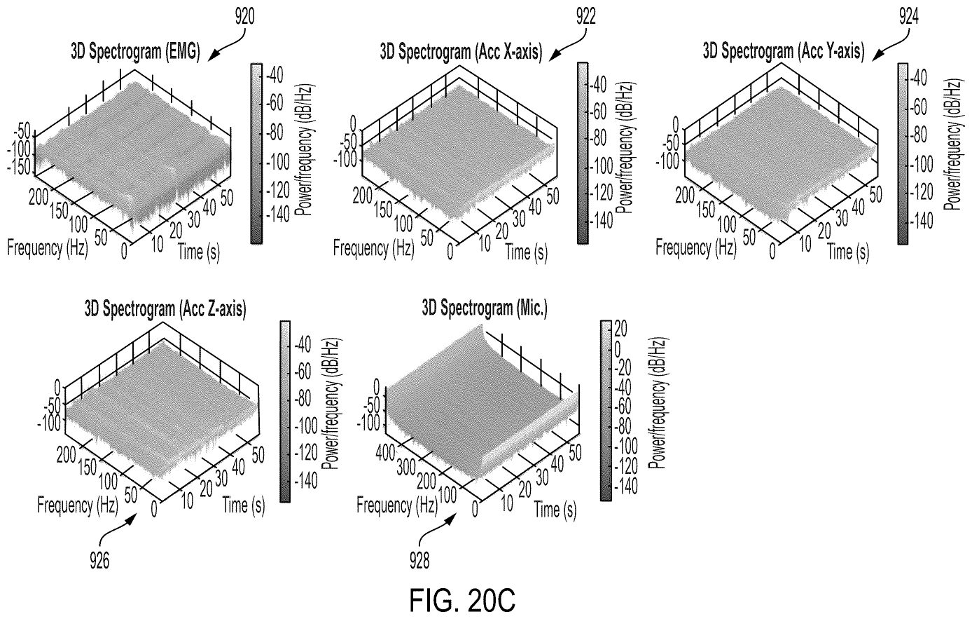

FIGS. 20A-20C illustrate the measured muscle activity and sound pressure waves over a 55 second drinking event. The thyrohyoid sEMG activity is provided at graph 900 (FIG. 20A), with a corresponding two-dimensional spectrogram provided at graph 910 (FIG. 20B) and a corresponding three-dimensional spectrogram provided at graph 920 (FIG. 20C). The measured X-axis acceleration is provided at graph 902 (FIG. 20A), with a corresponding two-dimensional spectrogram provided at graph 912 (FIG. 20B) and a corresponding three-dimensional spectrogram provided at graph 922 (FIG. 20C). The measured Y-axis acceleration is provided at graph 904 (FIG. 20A), with a corresponding two-dimensional spectrogram provided at graph 914 (FIG. 20B) and a corresponding three-dimensional spectrogram provided at graph 924 (FIG. 20C). The measured Z-axis acceleration is provided at graph 906 (FIG. 20A), with a corresponding two-dimensional spectrogram provided at graph 916 (FIG. 20B) and a corresponding three-dimensional spectrogram provided at graph 926 (FIG. 20C). The measured sound pressure waves are provided at graph 908 (FIG. 20A), with a corresponding two-dimensional spectrogram provided at graph 918 and a corresponding three-dimensional spectrogram provided at graph 928 (FIG. 20C).

As shown in graph 900, the sEMG signal varies in a distinct manner when the wearer is swallowing (i.e., compare swallowing points 905 of graph 906 compared with the corresponding time frames of graph 900), while the remaining time periods remain relatively near the baseline measured during the walking and stationary time periods. The measured accelerations along the X-axis and the Y-axis illustrated in each of the graphs 902 and 904, respectively, vary throughout the drinking event, while the acceleration along the Z-axis illustrates a distinct variance during swallowing points 803. The measured sound pressure waves illustrated in graph 908 also experience change throughout the drinking event.

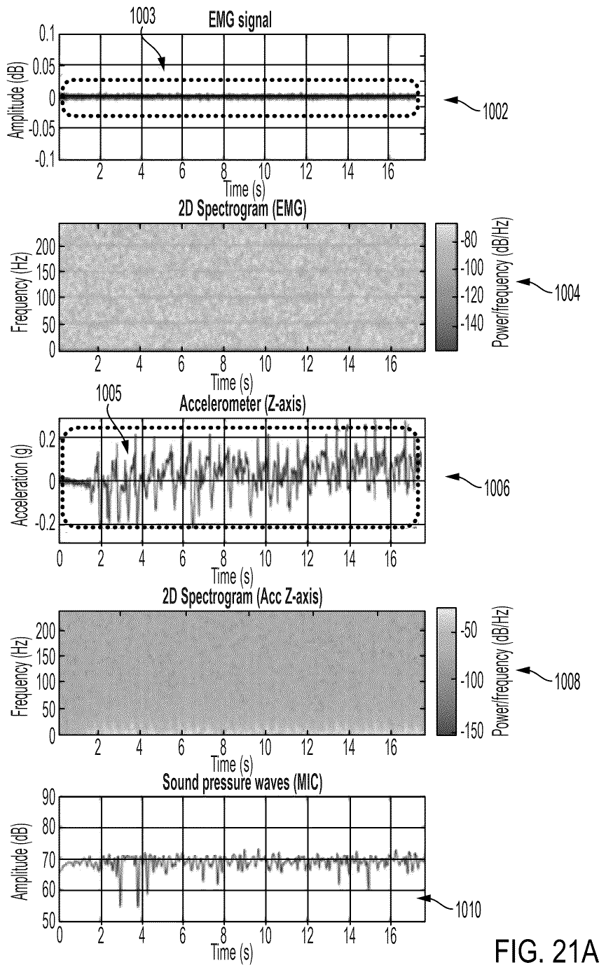

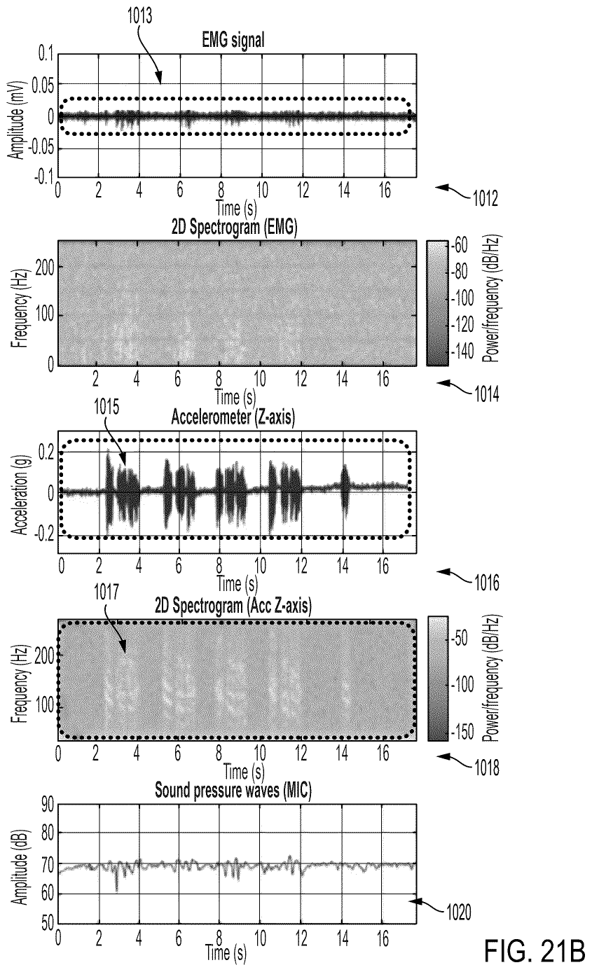

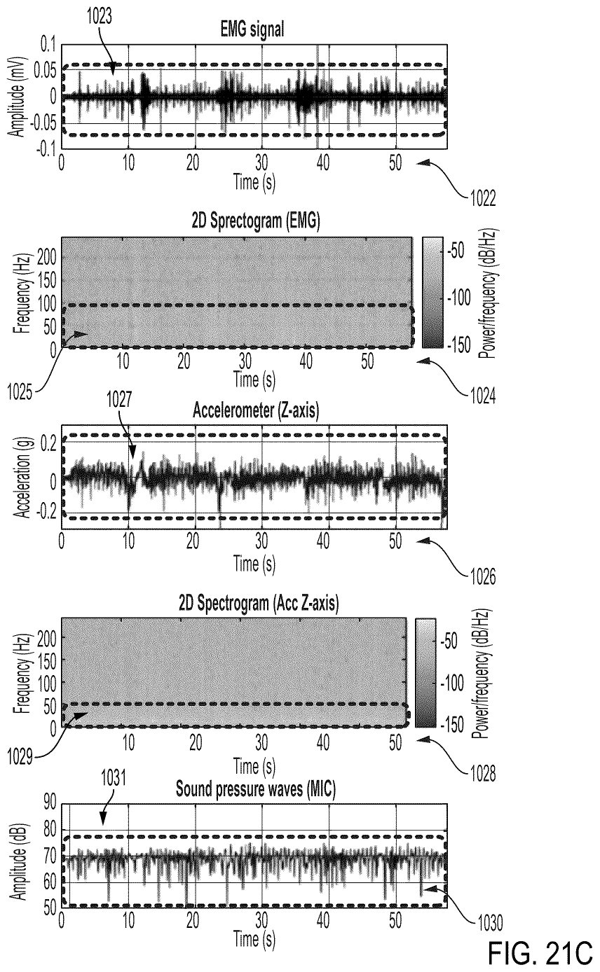

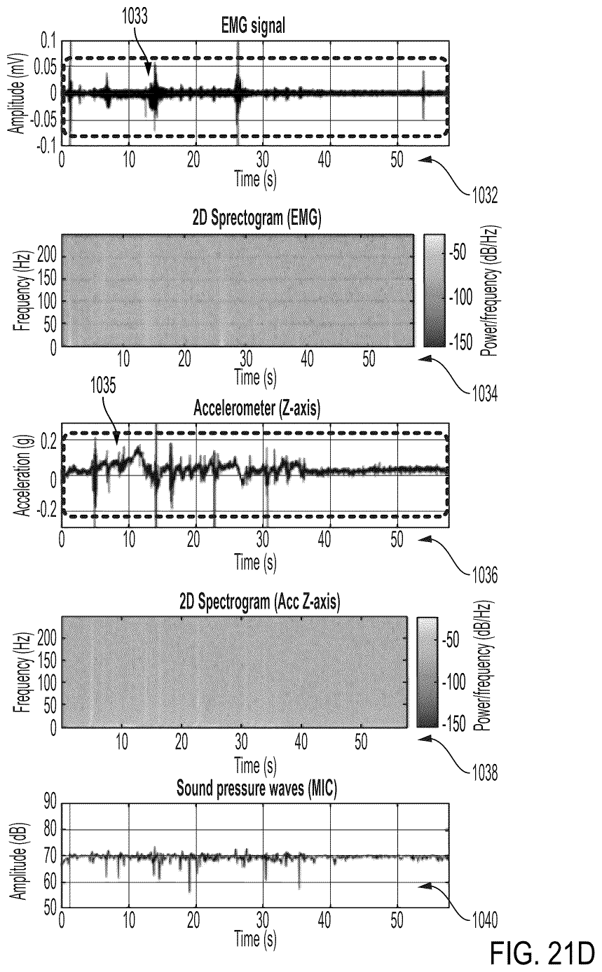

FIGS. 21A-21D illustrate distinguishing graphical features of measured muscle activity, acceleration, and sound pressure waves for walking, talking, swallowing, chewing, and drinking.

FIG. 21A illustrates the measured muscle activity, acceleration, and sound pressure waves over a 16 second walking event. The thyrohyoid sEMG activity is provided at graph 1002, with a corresponding two-dimensional spectrogram provided at graph 1004. The measured Z-axis acceleration is provided at graph 1006, with a corresponding two-dimensional spectrogram provided at graph 1008. The measured sound pressure waves are provided at graph 1010. As illustrated, a walking event can be distinguished (and eliminated) from talking, swallowing, chewing and drinking based on a flat sEMG signal 1003 and a high amplitude acceleration 1005 on the Z axis. Although not shown, a walking event may also exhibit high acceleration on the X and Y axes. By examining the graphical features of at least measured muscle activity and acceleration, a walking event may be distinguished from a swallowing event involving chewing or drinking.

FIG. 21B illustrates the measured muscle activity, acceleration, and sound pressure waves over a 16 second talking event. The thyrohyoid sEMG activity is provided at graph 1012, with a corresponding two-dimensional spectrogram provided at graph 1014. The measured Z-axis acceleration is provided at graph 1016, with a corresponding two-dimensional spectrogram provided at graph 1018. The measured sound pressure waves are provided at graph 1020. As illustrated, a talking event can be distinguished (and eliminated) from swallowing, chewing and drinking events based on a low amplitude sEMG signal 1013, a high amplitude acceleration 1015 on the Z axis (and the X-axis, not shown), and relatively high frequency content of the Z-axis acceleration 1017 compared to swallowing, chewing and drinking (compare features 1017 at graph 1018 with features 1029 at graph 1028, explained further below—this may also be true for the frequency content of X and Y axes acceleration, not shown). By examining the graphical features of at least measured muscle activity and acceleration, a talking event may be distinguished from a swallowing event involving chewing or drinking.

FIG. 21C illustrates the measured muscle activity, acceleration, and sound pressure waves over a 50 second chewing event. The thyrohyoid sEMG activity is provided at graph 1022, with a corresponding two-dimensional spectrogram provided at graph 1024. The measured Z-axis acceleration is provided at graph 1026, with a corresponding two-dimensional spectrogram provided at graph 1028. The measured sound pressure waves are provided at graph 1030. As illustrated, a chewing event can be distinguished from a walking or talking event based on a high amplitude sEMG signal 1023, a dominant low frequency EMG signal 1025 (compare with graphs 1004 and 1014 for the walking and talking events, respectively), a high amplitude acceleration 1027 on the Z axis (and the X and Y axes, not shown), a relatively low frequency content of the Z-axis acceleration 1029 compared to walking and talking events (compare features 1029 at graph 1028 with graphs 1008 and 1018 for walking and talking events, respectively—this may also be true for the X and Y axes, not shown), and high amplitude sound pressure waves 1031 detected by the microphone. By examining the graphical features of at least measured muscle activity, acceleration, and sound pressure waves, a chewing event may be distinguished from walking or talking events.

FIG. 21D illustrates the measured muscle activity, acceleration, and sound pressure waves over a 50 second drinking event. The thyrohyoid sEMG activity is provided at graph 1032, with a corresponding two-dimensional spectrogram provided at graph 1034. The measured Z-axis acceleration is provided at graph 1036, with a corresponding two-dimensional spectrogram provided at graph 1038. The measured sound pressure waves are provided at graph 1040. As illustrated, a drinking event can be distinguished from a chewing event based on periods of relatively high frequency content of the Z-axis acceleration compared to chewing (observe how graph 1038 for drinking exhibits higher frequency content at certain time points than graph 1028 for chewing events—this may also be true for the X and Y axes, not shown). Also, similar to chewing events, a drinking event can be distinguished from walking and talking events based on a high amplitude sEMG signal 1033. By examining the graphical features of at least measured muscle activity and acceleration, a drinking event may be distinguished.

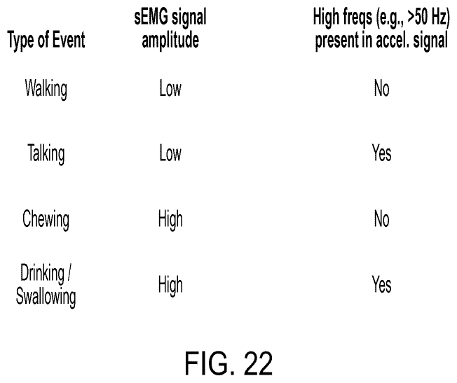

FIG. 22 presents a summary of the insights presented above through FIGS. 21A-D regarding how to distinguish between walking, talking, chewing, and drinking/swallowing events. Compared to chewing and drinking events, both walking and talking events are expected to exhibit lower sEMG signal amplitudes. The person of ordinary skill in the art would appreciate that, in any given implementation, the threshold between a “low” and a “high” sEMG signal amplitude will depend on the type and configuration of sEMG sensors. Also, when the accelerometer signals are analyzed in the frequency domain, both walking and chewing events are expected to be predominantly composed of low-frequency signals below approximately 50 Hz. Conversely, talking and drinking/swallowing events are expected to comprise at least some time periods with higher frequency signal content (e.g., greater than approximately 50 Hz). Although FIGS. 18A-D presented only spectrograms of Z-axis accelerations, accelerometer signals in the X- and Y-axes are also expected to exhibit similar behavior. These distinctions may be used to distinguish between walking, talking, chewing, and drinking/swallowing events.

### Example 20

Mechanical analysis was conducted using Finite Element Analysis (“FEA”) through a commercial software package, Abaqus, to investigate the stress and strain levels applied to the sensor module by folding, bending, stretching, and twisting, as well as the interfacial strain on human skin when the sensor module was attached. The developed sensor module was modeled, comprising a polyimide (PI) frame covered with an Ecoflex 00-35 body through which a textile line passed. Linear elasticity was applied to the PI frame, brass alloy electrode, and human skin with elastic moduli of 7.1 GPa, 97 GPa, and 10 kPa, and Poisson's ratios of 0.3, 0.31, and 0.48, respectively. A hyperfoam material model was applied to the textile based on uniaxial testing data. A Neo-Hookean hyperelastic model was applied to Ecoflex 00-35 with coefficients, C10 of 0.0113 and D1 of 1.96. Displacement and rotation boundary conditions were set for the edges of the PI frame, surfaces of Ecoflex 00-35, and the electrode. An embedded region constraint was applied to the PI frame, textile, and brass alloy electrode with Ecoflex 00-35 selected as the host material. Self-contact interaction was set for the textile, and surface contact interaction was set for the bottom surfaces of the brass alloy electrodes with the skin surface to analyze interfacial deformation. Subsequent to the computational work, an experimental study was conducted using a motorized force tester (ESM303, Mark-10), where the smart neckband was subjected to stretching conditions.

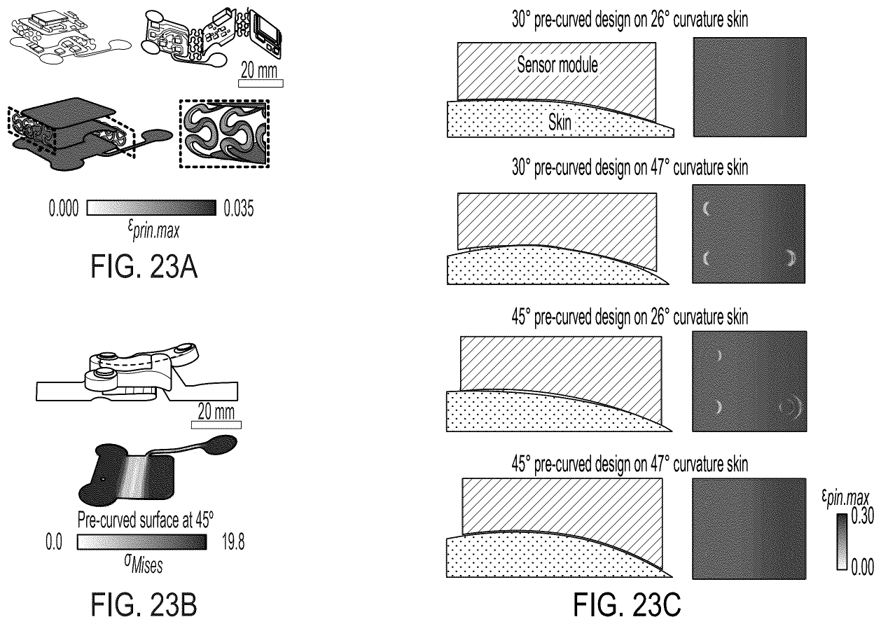

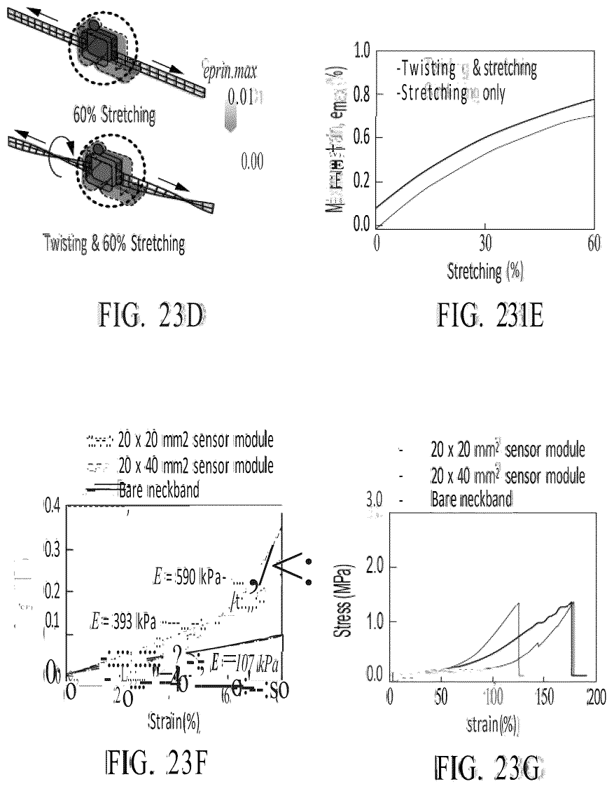

As shown in FIG. 23A, the sensor module placed in the stacked configuration experiences a maximum principal strain of less than 3.5% in the folded areas. FIG. 23B highlights the 45° pre-curved surface of the sensor module, which is designed to reconcile the curvature discrepancy between the module and the natural contour of the neck. Given that neck circumferences can vary widely—from 29.9 cm to 54.0 cm—a potential curvature gap of 26° to 47° may occur when aligning the sensor module with the skin. This gap could lead to bending stresses, resulting in user discomfort, the potential delamination of the device, or even skin irritation due to excessive pressure. FIG. 23C presents FEA results comparing sensor modules with 30° and 45° pre-curved surfaces. When applied to skin curvatures of 26° and 47°, the 30° pre-curved design exhibited principal strains of 6.5% and 30%, whereas the 45° pre-curved design exhibited strains of only 17% and 3%, respectively. Additional FEA data indicated that the sensor module with the 45° pre-curved design had a von Mises stress of 14.2 MPa and a minimal principal strain of 0.2%. Therefore, the 45° pre-curved design more closely follows the natural curvature of the neck, effectively reducing the curvature gap and improving mechanical compatibility with the skin.

FIG. 23D displays FEA results that illustrate the mechanical durability of the wearable device under conditions of 60% stretching and simultaneous twisting. These conditions take into account both the up to 29.3% strain experienced by the skin during dynamic activities and its potential for up to 15% stretch to accommodate variations in neck circumference. FIG. 23E presents the corresponding FEA results, which detail the maximum strain variations within the sensor module under these conditions. These findings indicate that the sensor module experiences less than 1% of the principal strain, highlighting its durability and appropriateness for practical use.

FIG. 23F provides a visual representation of the mechanical assessments for the sensor modules in two distinct sizes, 20×20 mm² and 20×40 mm², when subjected to a 60% stretch. The findings indicate that the smaller sensor module possesses a 393 kPa elastic modulus, roughly 50% less than the 590 kPa modulus of the larger one. Moreover, under identical stretching conditions, the smaller sensor module endured a stress of 147 kPa. In contrast, the larger sensor module and the bare neckband recorded stresses of 158 kPa and 72 kPa, respectively. FIG. 23G illustrates that, when subjected to strains of up to 120%, the larger sensor module experienced a more significant increase in stress compared to the smaller one. These observations highlight the mechanical benefits and stress resilience of miniaturized sensor modules, particularly in terms of stress alleviation within the smart neckband.

### Example 21

In analyzing the inherent vibrational response of the sensor module, a vibration generator (1000701, 3B Scientific) and an arbitrary waveform generator (3390, Keithley) was used to produce the targeted vibration. The waveform parameters were set to 2 Hz, square wave, 4 V_{Peak-Peak}, with an 80% duty cycle. To assess the waterproofing performance of the sensor module, it was repeatedly exposed to external water for 20 seconds. The sensor module was observed functioning normally under flowing water, successfully transmitting sensor data via Bluetooth®. To measure the pressure between the skin and the sensor module, both pre-curved and non-curved sensor modules were evaluated using a miniaturized pressure sensor (CSU8-1N, SingleTact). A high-resolution science grade long-wave infrared camera (A655sc, FLIR) to monitor temperature changes when the sensor module was operational on the skin for 18 hours.

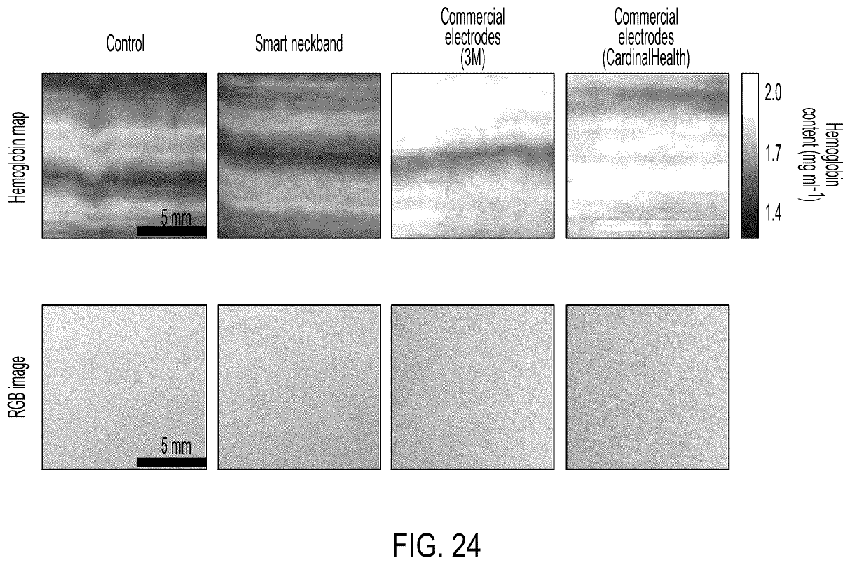

Inflammation accompanied by erythema, which changes the concentrations of hemoglobin, is a common response after irritation of the human skin. A line-scan hyperspectral imaging system was used to quantify the inflammation caused by irritants. The hyperspectral imaging system was used to acquire a hyperspectral image (hypercube) of a sample to analyze spectral fingerprints from a specific area. The line-scan hyperspectral imaging system used has a slit that had a width of 23 μm. The light passing through the slit was dispersed by a diffraction grating (groove density=150 mm⁻¹) and captured using a monochrome camera (GS3-U3-120S6M-C, FLIR). An LED light source with a color temperature of 6500K (D65) was used as the illumination source. Spectral calibration of the spectrograph was performed using a xenon calibration light source that emits multiple narrow peaks at specific wavelengths. A fixed focal length lens (MVL25M1, Navitar) was used mainly to image the skin area and the field of view was as small as 10 mm×10 mm. The same area was imaged with a smartphone camera (iPhone 11 Pro, Apple) to capture RGB images. The tested wearable device was applied on the inside of the human forearm (medial antebrachial cutaneous of the forearm) for 2 hours. As positive controls, 3M electrode (2560, 3M) and CardinalHealth electrode (H124SG, CardinalHealth), were attached on the same area for 2 hours. Images were acquired before and after the experiment for hemoglobin contents comparison. A mechanical linear scan step was performed at 0.25 mm. The data was acquired using a custom-built MATLAB interface. A tissue reflectance spectral model was used to extract key hemodynamic parameters from the ground-truth hyperspectral image. Light propagation in tissue can be modeled in accordance with the theory of radiative transport and robust approximations (e.g., diffusion, Born, and empirical modeling). Specifically, parameter extractions were conducted using an extensively used empirical modeling method. The intensity reflected from a biological sample can be expressed as a function of λ in the visible range:

$${I}_{R} ( λ ) = [ {b}_{1} {( \frac{λ}{{λ}_{0}} )}^{{b}_{2}} + {b}_{3} {( \frac{λ}{{λ}_{0}} )}^{- 4} ] exp [ - {b}_{4} \times \{ {b}_{5} \times {ε}_{{HbO}_{2}} ( λ ) + ( 1 - {b}_{5} ) \times {ε}_{Hb} ( λ ) \} ]$$

where b₁, b₂, and b₃ are associated with the scattering (Mie or Rayleigh) contributions at λ₀=800 nm, ε_{HbO}_{₂}(λ) denotes the absorption coefficient of oxygenated hemoglobin (HbO2), ε_{Hb}(λ) denotes the absorption coefficient of deoxygenated hemoglobin (Hb), b₄ is the hemoglobin concentration multiplied by the optical pathlength, and b₅ is the blood oxygen saturation (sPO2). The hemoglobin contents multiplied by the optical pathlength (b₄) was used to indicate the level of skin irritation as shown in FIG. 24. All fitting parameters were computed using the simplex search (Nelder-Mead) algorithm.

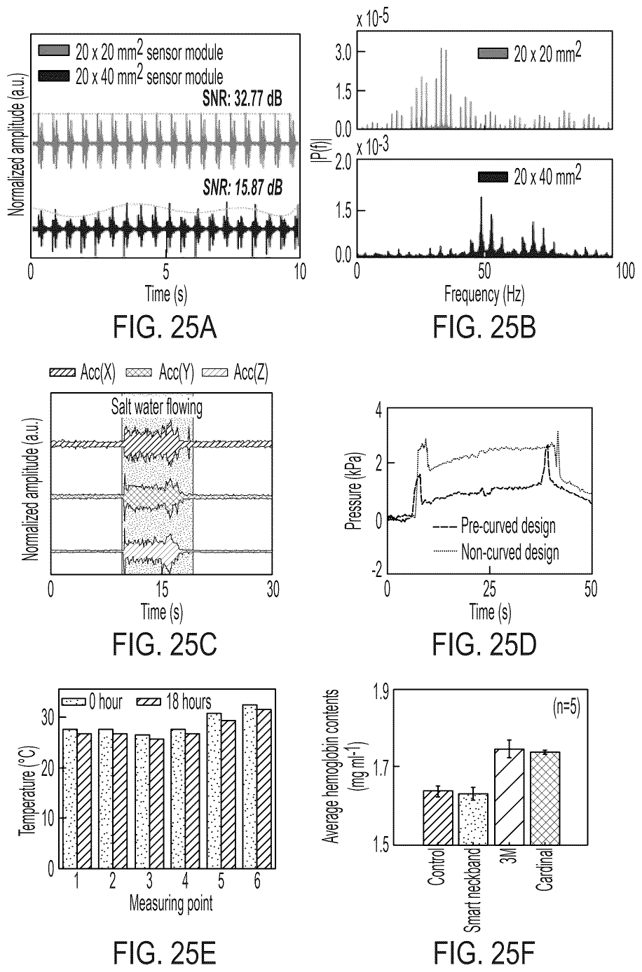

To assess the performance of the tested wearable device in practical uses, comprehensive benchtop tests were conducted to evaluate its response to factors such as vibration, water exposure, and temperature fluctuations. The focus of the test included typical sub-2 Hz natural vibration frequency of the human body while walking to analyze the signal characteristics from the sensor module under a 2 Hz vibration. FIG. 25A illustrates that the smaller sensor module (20×20 mm²) maintained a consistent vibration recording, in stark contrast to the larger module (20×40 mm²), which displayed irregular waveforms. The SNR of the smaller module was 32.77 dB, a figure substantially higher than the 15.87 dB recorded for the larger module. FIG. 25B presents further insights from a fast Fourier transform (FFT) analysis, revealing a clear 2 Hz signal from the smaller module compared to the substantial noise exhibited by the larger module. The variations in the mechanical resonance frequencies were also notable: around 30 Hz for the smaller module and 50 Hz for the larger module. These variations are attributable to the changes in physical properties as the overall size of the module increases, including a weight increase of 87% from 12.03 g to 22.52 g.

Waterproofing tests were conducted to verify the sensor module's resilience to potential sweat exposure during use. The results, shown in FIG. 25C, demonstrate its sustained functionality under a continuous flow of salt water. These tests underscore its reliability in scenarios prone to induce sweat, such as long-duration usage, engaging in physical activity, and exposure to high environmental temperatures. Potential pressure-related risks were also tested, notably the danger of tissue ischemia which could occur if the pressure between the sensor and the skin exceeds 4.3 MPa. Testing demonstrated that the wearable device maintains pressures significantly below this threshold—0.97 kPa and 2.49 kPa for the pre-curved and non-curved sensor modules respectively (FIG. 25D)—highlighting the ischemia-safe nature of the wearable device, with the pre-curved format further minimizing skin pressure.

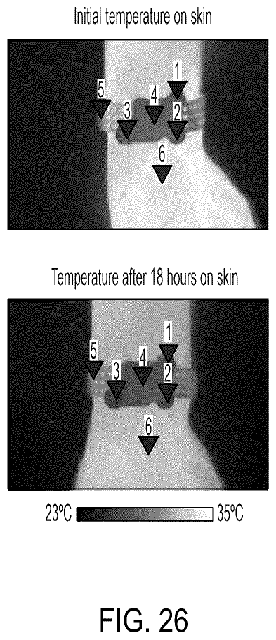

Assessments revealed that the wearable device has a maximum temperature increase of just 1.4 °C over 18 hours of continuous wear, with temperatures not exceeding 31.7 °C (FIG. 25E), well below the threshold temperature of above 44 °C, at which low-temperature burns may occur. FIG. 25F shows the measurement results of average hemoglobin contents of the skin. FIG. 26 offers a detailed visualization of temperature variations on both the sensor module surface and the adjacent skin areas over the 18-hour period.

### Example 22

This study involved healthy volunteers at Purdue University. The participant pool consisted of 6 volunteers, with an age range of 29-34 years and an equal distribution of male and female subjects. Selection criteria were as follows: (1) being aged between 18 and 55 years, (2) the capacity to understand and give informed consent, and (3) a willingness to fully participate in the study procedures, including wearing the wearable device for the activity-rest cycles. Prior to the main procedures, participants attended an orientation session to familiarize themselves with the study's expectations. An eligibility survey was administered to determine if participants met the study criteria. Those who qualified received a comprehensive consent form outlining the study's objectives, participant responsibilities, and ethical considerations, such as potential risks and benefits. Before wearing the wearable device, participants were briefed on its purpose and functionality. The sensor module was fitted on each participant's thyrohyoid muscle. The experimental protocol consisted of alternating cycles of 20-second rest and 20-second activity intervals, repeated 20 times for an approximate total of 13 minutes. This cycle was conducted four times, each for different activities: body movement, fluid intake, food intake, and speech. Thus, each session took an estimated 54 minutes to complete. The above procedure was carried out under two conditions: stationary and active (walking at a comfortable pace), making the total duration approximately 108 minutes per session.

All analyses were conducted using the MATLAB (R2021b) technical computing language. The digital manipulation phase involved the use of a fourth-order Butterworth infinite impulse response filter, which was followed by an anti-causal, zero-phase filtering approach implemented through the MATLAB ‘filtfilt’ function. To illustrate the distribution of signal frequencies over time, spectrogram analysis was completed using the MATLAB ‘pspectrum’ function. Moreover, FFT analysis was executed using the MATLAB ‘FFT’ function to identify the individual periodic components of the signal, aiding in a deeper comprehension of the signal characteristics. Exploring the details of signal processing, it was observed that body movements were characterized by substantial impact forces that spanned a wide frequency range, reaching up to around 100 Hz. The onset of swallowing events was marked by slow movements (~0.1 s) of the vocal folds and larynx mechanics during the pharyngeal phase. This phase ended with a high-frequency ringdown associated with the flow of fluid or food during the esophageal phase. The swallowing events encompassed both low-frequency mechanical motions (0.1-5 Hz) and high-frequency acoustic components (100-800 Hz). We also noted the complexity of mastication biomechanics, where the mandibular cycle typically exhibited signal frequencies in the 1-2 Hz range. Speech signals were distinct, displaying rich harmonic structures with fundamental frequencies that generally ranged between 85 and 255 Hz for the adult population.

An RF classifier, as described further in Example 23, was employed to automatically identify a range of activities. This tool constructs multiple decision trees from different subsets of the dataset and amalgamates their predictions to determine a final outcome. Its versatility allows it to detect complex patterns in the data, rendering it ideal for multi-faceted classification tasks. The RF classifier was fine-tuned using k-fold cross-validation (CV), wherein 90% of the data was allocated for training and the remaining 10% for testing, a process repeated in ten cycles. The classification results were then averaged across these iterations.

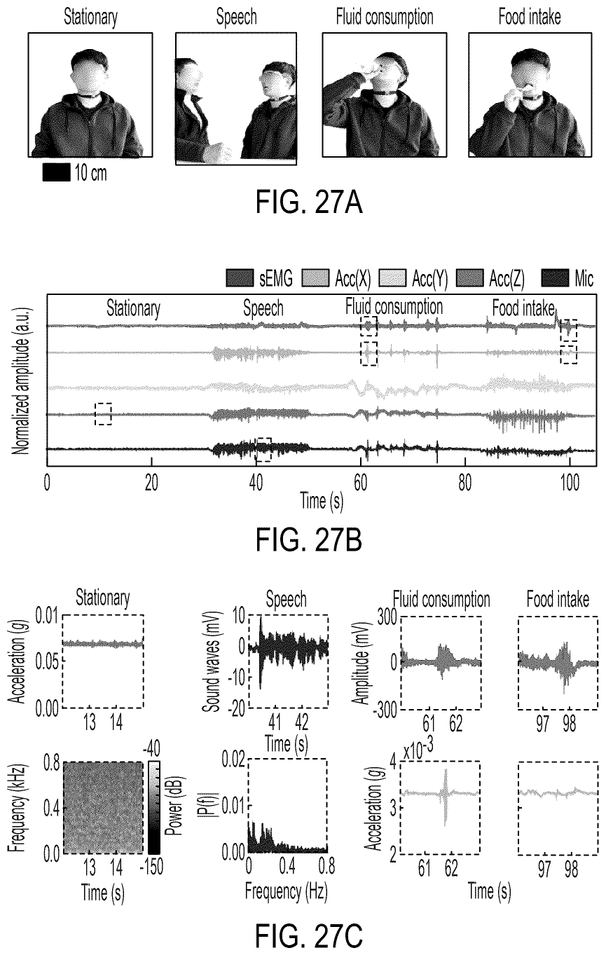

FIG. 27A depicts a stationary experiment scenario where a healthy 32-year-old subject, consuming 17 oz of water and potato chips, engages in activities including speaking, and fluid consumption and food intake. FIG. 27B delineates the signals recorded from various sensors—including the sEMG amplifier, a three-axis accelerometer, and a microphone—strategically positioned on the thyrohyoid muscle. These signals capture an initial 5-second control state, succeeded by a 20-second stationary state before the initiation of each activity. Further data, including details on speech, fluid consumption, and food intake, were amassed in 5-second intervals. These readings vividly portray unique features in both time and frequency domain analyses, providing a rich data set concerning dietary habits during stationary phases. FIG. 27C presents a granular view of the specific signal patterns associated with distinct physiological events. In a stationary state, subtle amplitude signals approximately in the range of ~10⁻² g were documented on the z-axis of acceleration. Addressing speech dynamics, pronounced amplitude signals were observable in sound recordings, with the FFT of these signals showcasing a heightened intensity in the 85-255 Hz frequency band, a characteristic markedly absent in other recorded activities. Delving into the swallowing activity during fluid consumption, a burst in signal patterns was noted in both the sEMG and x-axis of acceleration, offering a two-pronged insight; the sEMG captured the thyrohyoid muscle contraction while the x-axis logged muscle movements occurring through the swallowing process, furnishing a valuable differentiation metric for swallowing activities. Furthermore, the mastication process preceding food intake registered lower amplitude movements in the sEMG, paving the way to a higher frequency ring-down associated with swallowing. This stage marked a distinct shift in the activity spectrum.

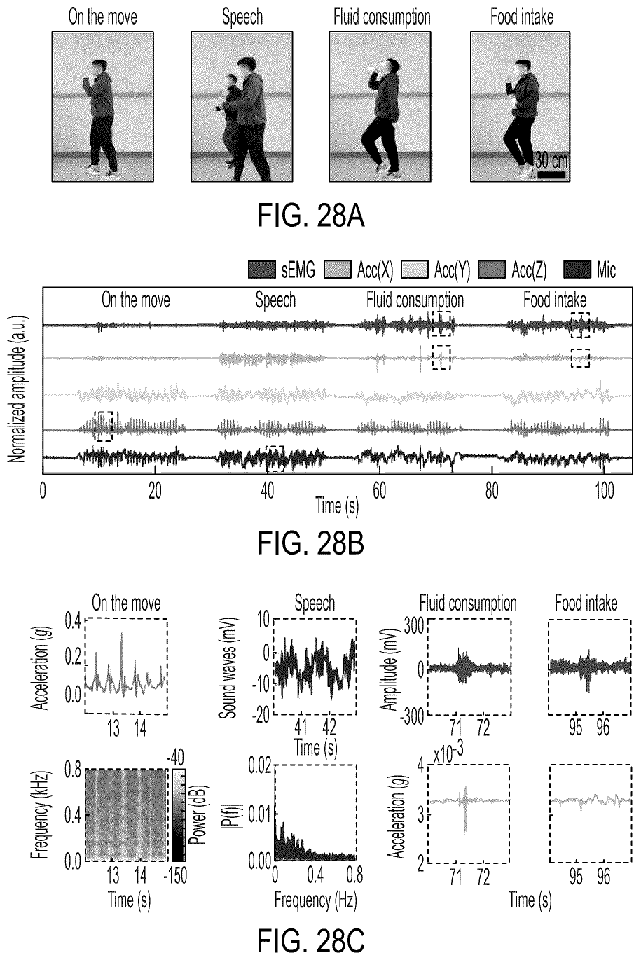

FIG. 28A depicts the experimental setup where the subject was engaged in activities such as speaking, consuming liquids, and consuming solids while walking around with a steady stride. The activities were carried out under conditions meticulously controlled to mirror those when the subject was stationary, accounting for factors such as the age of the subject, the type of liquid consumed, and the food ingested. FIG. 28B showcases the data captured from the sEMG amplifier, the tri-axial accelerometer, and the microphone during this period. This phase of the experiment followed the same sequence as its stationary counterpart. FIG. 28C reveals the specific quantitative attributes of the signal patterns associated with individual physiological events during these activities. Examining the z-axis acceleration while the subject was active, signals with larger amplitudes were noted, approximately in the region of ~4×10⁻¹ g. The spectrogram analysis corroborates the periodic presence of high-intensity, high-frequency signals under active conditions, enhancing understanding of body dynamics and dietary habits during movement. Regarding speech activities, there were substantial amplitude variations in the sound signals. Moreover, FFT analyses revealed a higher concentration of intensity within the 85-255 Hz bandwidth compared to other engagements. A notable observation was the marked increase in power within the lower frequency band during active states compared to stationary phases, a phenomenon likely influenced by movement. Focusing on swallowing during fluid consumption, a burst pattern in both the sEMG and x-axis acceleration readings was observed, segmented into two unique parts. The first highlighted the contraction of the thyrohyoid muscle, a detail captured by the sEMG, while the second, recorded in the x-axis acceleration, showcased the muscle movements occurring throughout the swallowing phase. This distinct signal profile does not appear in data concerning general bodily motions and speech, facilitating precise identification of swallowing maneuvers. When analyzing mastication associated with food intake, initial small-amplitude masticatory actions were detectable in the sEMG readings before the higher frequency ringdown observed during swallowing. Importantly, despite the minimal signal magnitudes experienced while attached to the neck, all these signals maintained high reliability, exhibiting SNR values of 13.18 dB for sEMG, 17.18 dB for x-axis acceleration, 12.21 dB for y-axis acceleration, 15.80 dB for z-axis acceleration, and 13.27 dB for the microphone.

### Example 23

The physiological signals harvested from the wearable device were initially divided into several fixed-length frames to facilitate activity prediction. Once normalized, the signals stemming from the same time instance were amalgamated horizontally to create feature vectors associated with specific activities. In the realm of individual activity detection, a single activity was pinpointed within a given frame, necessitating a multi-class approach whereby a machine learning (ML) or deep learning (DL) algorithm would discern one class from a plethora of others. Conversely, concurrent activities presented a scenario wherein multiple activities coexisted in a solitary data frame. This condition dictated a shift from identifying single activities to predicting each unique activity present in the frame, a strategy realized through the utilization of the Label Powerset algorithm. This tool transformed the multi-class dataset into a multi-label format, a transition that retained label correlation while curtailing the probability of spawning unrealistic predictions. Subsequently, the reconfigured multi-class data were introduced to an ML algorithm known as the RF classifier, setting the stage for model fitting and predictive analysis.

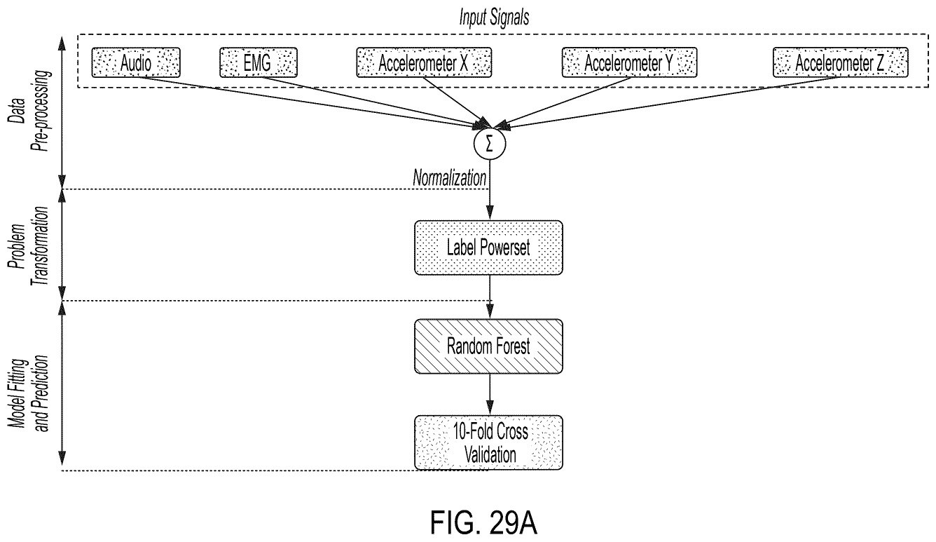

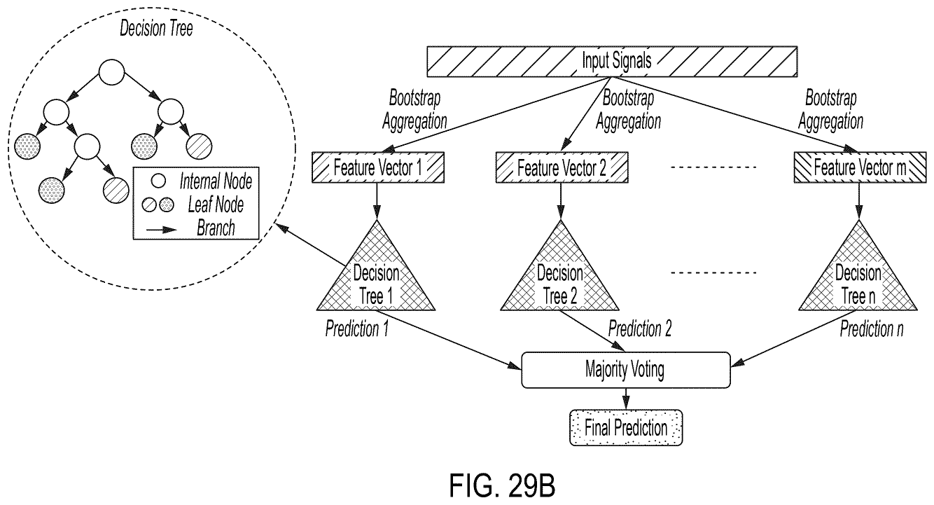

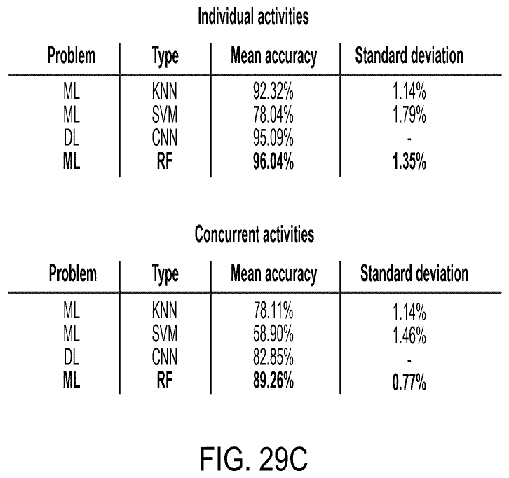

The training dataset encompassed a spectrum of activities—individual instances (encompassing stationary, active, speech, fluid consumption, and food intake) and concurrent permutations (featuring combinations of active states with speech, fluid consumption, and food intake). A comprehensive illustration of the activity recognition approach is shown in FIG. 29A. Furthering the predictive endeavor, Classification and Regression Tree (CART) classifiers were deployed to forecast either a distinct class or a target value, drawing upon a decision tree algorithm. The RF is a combination of these CARTs that are denoted as Tn:

T=T₁(X),T₂(X), . . . ,Tₙ(X)

Where:

X=[x₁,x₂, . . . ,xₘ]

is an m-dimensional feature vector. The RF was acknowledged as a supremely precise ML algorithm, leveraging a randomly chosen assortment of inputs at each node to formulate a preset array of decision trees. Employing bootstrap aggregation, the RF constructed divergent training subsets from the training samples, each maintaining an identical sample quantity per node, and facilitated a democratic process where each tree endorsed the prevalent class within its purview. In this scenario, every decision tree drew upon a unique sub-dataset to fashion a prediction. A concurrence emerged through a majority ruling orchestrated by the RF, a process meticulously detailed in FIG. 29B. Here, a preset m number of inputs were haphazardly utilized for the nodes, instigating a series of votes for the most likely class and culminating in a collection of n votes. These inputs, distinguished by a color gradient for each feature vector, facilitated the spontaneous generation of sub-datasets. Following this, the redefined problem set derived from the preceding stage found its way to the RF classifier, a move that yielded activity predictions ripe for performance evaluation. The simulation parameters were firmly set with a tree count of 50 and a two-second frame duration. Furthermore, a comparative analysis of the RF classifier was instituted against alternative ML strategies and a traditional CNN-based DL blueprint. The RF classifier outperformed the CNN paradigm and rival techniques including k-nearest neighbors (KNN) and support vector machine (SVM), underscoring its adeptness in tackling complex classification undertakings (FIG. 29C). In pursuit of impeccable accuracy, we initiated every simulation bar the DL route, a total of ten times, deploying a 10-fold CV to endorse the results steadfastly. The experiments were conducted on NVIDIA Geforce GTX 1080 Ti.

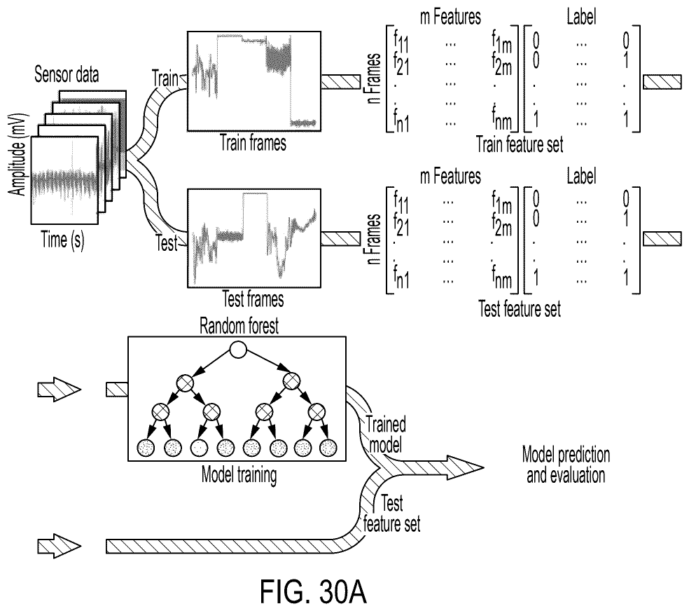

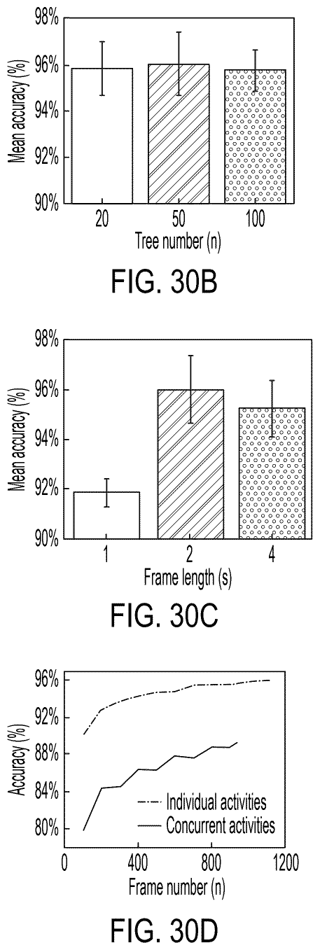

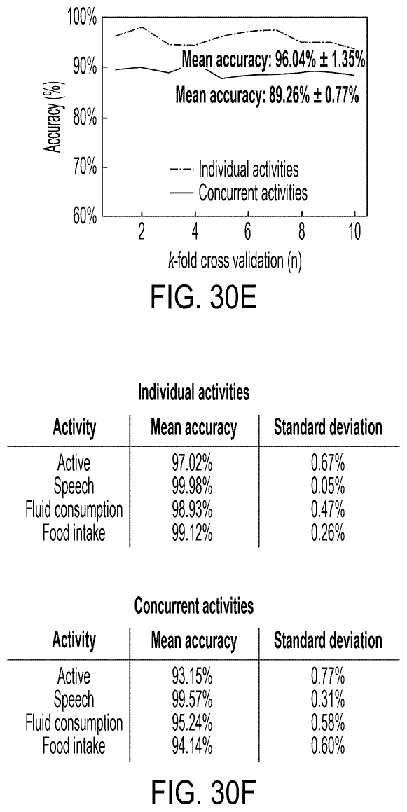

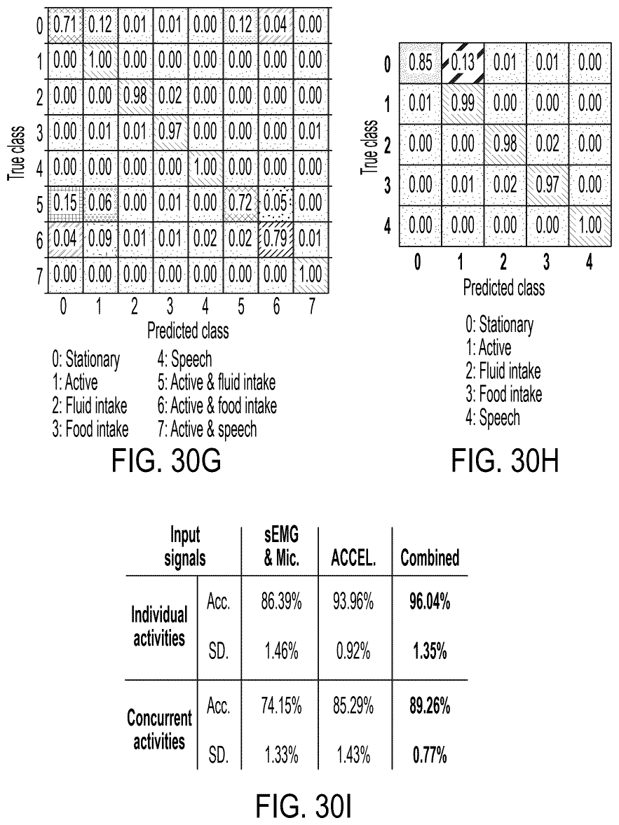

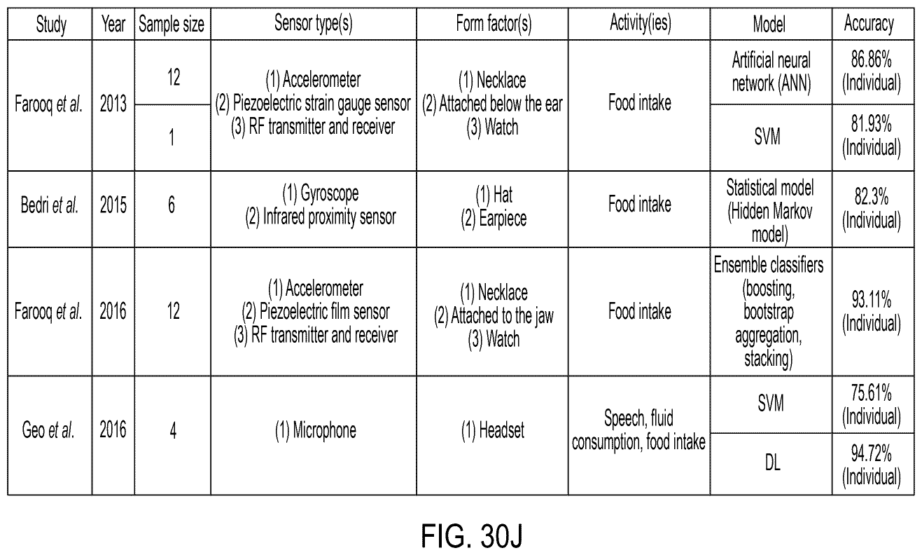

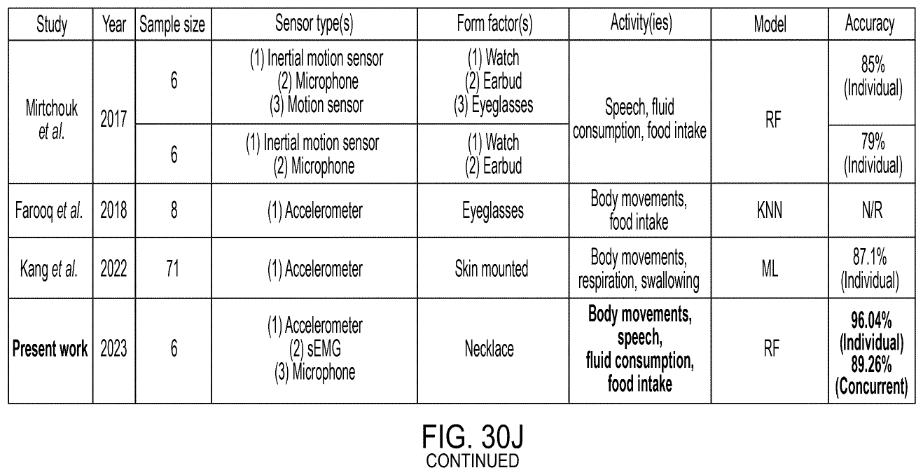

FIG. 30A outlines the signal processing pipeline and highlights the critical role the optimization of RF classifier parameters. This optimization scrutinized two key parameters: the number of trees in the forest and the frame length, which corresponds to the data capture time window. The number of trees profoundly affects the classifier's efficiency; a forest that is too sparse might overlook essential data patterns, while an overly dense one can risk overfitting, compromising performance on unseen data. Through careful calibration using k-fold cross-validation (CV) and analyzing between 20 and 100 trees, it was determined that a forest of 50 trees offers the optimal balance for peak performance (FIG. 30B). Frame length emerged as another pivotal parameter, with its choice being directly consequential to the amount of information available for classification. A 2-second frame was identified as optimal (FIG. 30C), skillfully avoiding the issues of information scarcity seen with shorter frames and the unnecessary complexity longer frames introduce without substantial performance improvement.

Additional fine-tuning was achieved by altering the frame count to assess the classifier's responsiveness to dataset size, finding the best results with 1,120 frames for individual activities and 940 for concurrent activities (FIG. 30D). Equipped with a refined RF classifier, utilizing 50 trees and a 2-second frame length, a 10-fold CV was conducted on a substantial, healthy training dataset to assess its effectiveness. This classifier, applied to data gathered from subjects performing a wide range of tasks, including both individual and concurrent activities, established a stringent benchmark for the system. The assessment metrics—subset accuracy and standard deviation, which measure the precise alignment of predicted and true labels and the classifier's consistency, respectively—revealed an impressive average accuracy rate of 96.04% for individual activities and 89.26% for concurrent activities (FIGS. 30E, 30F).

FIG. 30G displays the confusion matrix, offering a detailed account of the accuracy attained for each activity during concurrent activities. A counterpart matrix for individual activities is shown in FIG. 30H. These matrices affirm the high precision with which the disclosed model can categorize both complex individual and concurrent activities. The analysis was extended to evaluate the impact of various input signals on the efficiency of the model, which leveraged inputs including audio, sEMG, and data from a three-axis accelerometer. Notably, the accelerometer data emerged as a pivotal factor in predicting both singular and concurrent activities, underlining its central role in activity recognition (FIG. 30I). This underscores the significance of integrating audio, sEMG, and accelerometer data to boost the performance of activity recognition systems. A comparative analysis was conducted and delineated in FIG. 30J.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

Various aspects are described in this disclosure, which include, but are not limited to, the following aspects:
(1) A wearable device, comprising: a textile; and a sensor system affixed to the textile, the sensor system comprising: at least two sEMG electrodes; an accelerometer; a circuit signal amplifier; a microcontroller; and a memory; wherein the at least two sEMG electrodes and the accelerometer of the sensor system are configured to be positioned over a thyrohyoid muscle of a human subject, and wherein the wearable device is configured for measuring data associated with underlying thyrohyoid muscle activity suitable for detecting a physical event of the human subject.
(2) The wearable device of aspect 1, wherein the wearable device is configured to be placed around a neck of the human subject.
(3) The wearable device of any of aspects 1-2, wherein the sensor system stores the measured data based on a timestamp.
(4) The wearable device of aspect 3, wherein the sensor system transmits the stored measured data to a remote computing device for processing to detect the physical event of the human subject.
(5) The wearable device of any of aspects 1-4, wherein the sensor system includes: a first segment, a second segment, and a third segment.
(6) The wearable device of aspect 5, wherein: the first segment houses the at least two sEMG electrodes and the accelerometer; the second segment houses the circuit signal amplifier; and the third segment houses the microcontroller and the memory.
(7) The wearable device of any of aspects 5-6, further comprising: a first bridge section coupling the first segment to the second segment; and a second bridge section coupling the second segment to the third segment.
(8) The wearable device of aspect 7, wherein the first bridge section and the second bridge section are foldable such that the first segment, the second segment, and the third segment are configured to be stackable to form a stacked configuration.
(9) The wearable device of aspect 8, wherein the textile is positioned between the first segment and the second segment when in a stacked configuration.
(10) The wearable device of any of aspects 8 and 9, wherein the first segment, the second segment, and the third segment are encapsulated by a polymer in the stacked configuration.
(11) The wearable device of aspect 10, wherein the polymer is an elastomer.
(12) The wearable device of any of aspects 5-11, wherein the first segment, the second segment, and the third segment are each 20 cm×20 cm.
(13) The wearable device of any of aspects 1-12 further comprising: a base layer and a cover associated with the sensor system.
(14) The wearable device of any of aspects 1-13, the sensor system further comprising a power source and an analog to digital converter.
(15) The wearable device of any of aspects 1-14, wherein the memory is removable.
(16) The wearable device of any of aspects 1-15, wherein the physical event comprises at least one of a food consumption event and a drink consumption event.
(17) The wearable device of any of aspects 1-16, wherein the physical event comprises an emesis event.
(18) The wearable device of any of aspects 1-17, wherein the sensor system comprises a microphone.
(19) The wearable device of any of aspects 1-18, wherein the textile is a mesh material.
(20) The wearable device of any of aspects 1-19, wherein the sensor system defines 45° precurved surface.
(21) A consumption journaling system, comprising: a wearable device having a sensor system comprising: at least two sEMG electrodes; an accelerometer; and a circuit signal amplifier; wherein the sensor system is configured to measure underlying muscle activity when the wearable device is positioned on a neck of a human subject; and a processor configured to analyze the measured underlying muscle activity to detect and distinguish between a solid food consumption event and a drink consumption event and log the solid food consumption event or the drink consumption event when detected.
(22) The consumption journaling system of aspect 21, wherein the processor is configured to implement a machine-learning algorithm to automatically detect and distinguish between the solid food consumption event and the drink consumption event based on the measured underlying muscle activity.
(23) The consumption journaling system of any of aspects 21-22, wherein the processor is configured to further detect and distinguish between non-food consumption events and food consumption events.
(24) The consumption journaling system of any of aspects 21-23, wherein the wearable device further comprises a removable memory.
(25) The consumption journaling system of any of aspects 21-24, wherein the processor is mounted on the wearable device.
(26) The consumption journaling system of any of aspects 21-24, wherein the processor is mounted on a remote computing device.
(27) A method for creating a consumption timeline, the method comprising: positioning a wearable device on a thyrohyoid muscle of a human subject; measuring thyrohyoid muscle activity with a plurality of sensors of the wearable device; processing the measured thyrohyoid muscle activity; identifying a physical event according to the processed thyrohyoid muscle activity, wherein the physical event is differentiated as one of a food consumption event and a non-food consumption event; and recording the physical event according to a timeline when the physical event is a food consumption event.
(28) The method of aspect 27, wherein a non-food consumption event includes at least one of remaining stationary, walking, and talking.
(29) The method of any of aspects 27-28, wherein the step of identifying the physical event includes automatically identifying the physical event according to a machine-learning algorithm.
(30) The method of any of aspects 27-29, wherein the sensors include at least two sEMG recording electrodes and an accelerometer.
(31) The method of any of aspects 27-30, wherein the step of processing the measured thyrohyoid muscle activity is implemented at a processor on the wearable device.
(32) The method of any of aspects 27-30, wherein the step of identifying the physical event is implemented at a processor on the wearable device.
(33) The method of any of aspects 27-30, wherein the step of identifying the physical event is implemented at a processor on a remote computing device.
(34) The method of any of aspects 27-33, further comprising determining at least one of a recommended dosage amount and a recommended dosage time for an administration of insulin based on the recorded physical event.
(35) The method of aspect 34, further comprising alerting a user of the at least one of the dosage amount and the dosage time.
(36) The method of aspect 34, further comprising instructing an insulin delivery device to administer insulin based on the recommended dosage amount or the recommended dosage time.
(37) A wearable device, comprising: a textile; and a sensor system, comprising: a first segment including a microphone, an accelerometer, and an sEMG electrode; a second segment including a charging circuit; a third segment including a microcontroller; a first bridge portion connecting the first segment and the second segment; and a second bridge portion connecting the second segment and the third segment.
(38) The wearable device of aspect 37, wherein each of the first bridge portion and the second bridge portion is defined by a plurality of serpentine interconnectors.
(39) The wearable device of any of aspects 37 and 38, wherein the first bridge portion and the second bridge portion are foldable such that the first segment, the second segment, and the third segment are configured to be placed in a stacked configuration.
(40) The wearable device of aspect 39, wherein the sensor system is encapsulated in the stacked configuration.
(41) The wearable device of aspect 40, wherein the sensor system is encapsulated in an elastomer material.
(42) The wearable device of any of aspects 40 and 41, wherein the textile is positioned between the first segment and the second segment in the stacked configuration.
(43) The wearable device of any of aspects 37-42, wherein the sensor system defines a 45° precurved surface.
(44) The wearable device of any of aspects 37-43, wherein the accelerometer is a three-axis accelerometer.
(45) The wearable device of any of aspects 37-44, wherein the sensor system is configured to be positioned over a thyrohyoid muscle of a human subject.

1. A wearable device, comprising:
a textile; and
a sensor system affixed to the textile, the sensor system comprising:
at least two sEMG electrodes;
an accelerometer;
a circuit signal amplifier;
a microcontroller; and
a memory;
wherein the at least two sEMG electrodes and the accelerometer of the sensor system are configured to be positioned over a thyrohyoid muscle of a human subject, and wherein the wearable device is configured for measuring data associated with underlying thyrohyoid muscle activity suitable for detecting a physical event of the human subject.

2. The wearable device of claim 1, wherein the wearable device is configured to be placed around a neck of the human subject.

3. The wearable device of claim 1, wherein the sensor system stores the measured data based on a timestamp.

4. The wearable device of claim 3, wherein the sensor system transmits the stored measured data to a remote computing device for processing to detect the physical event of the human subject.

5. The wearable device of claim 1, wherein the sensor system includes:
a first segment, a second segment, and a third segment.

6. The wearable device of claim 5, wherein:
the first segment houses the at least two sEMG electrodes and the accelerometer;
the second segment houses the circuit signal amplifier; and
the third segment houses the microcontroller and the memory.

7. The wearable device of claim 5, further comprising:
a first bridge section coupling the first segment to the second segment; and
a second bridge section coupling the second segment to the third segment.

8. The wearable device of claim 7, wherein the first bridge section and the second bridge section are foldable such that the first segment, the second segment, and the third segment are configured to be stackable to form a stacked configuration.

9. The wearable device of claim 8, wherein the textile is positioned between the first segment and the second segment when in a stacked configuration.

10. The wearable device of claim 8, wherein the first segment, the second segment, and the third segment are encapsulated by a polymer in the stacked configuration.

11. The wearable device of claim 10, wherein the polymer is an elastomer.

12. The wearable device of claim 5, wherein the first segment, the second segment, and the third segment are each 20 cm×20 cm.

13. The wearable device of claim 1, further comprising: a base layer and a cover associated with the sensor system.

14. The wearable device of claim 1, the sensor system further comprising a power source and an analog to digital converter.

15. The wearable device of claim 1, wherein the memory is removable.

16. The wearable device of claim 1, wherein the physical event comprises at least one of a food consumption event and a drink consumption event.

17. The wearable device of claim 1, wherein the physical event comprises an emesis event.

18. The wearable device of claim 1, wherein the sensor system comprises a microphone.

19. The wearable device of claim 1, wherein the textile is a mesh material.

20. The wearable device of claim 1, wherein the sensor system defines a 45° precurved surface.

21-45. (canceled)
